(19) United States
(12) Patent Application Publication
(10) Pub. No.: US 2018/0214430 A1
(43) Pub. Date: Aug. 2, 2018

(54) Selective Inhibitors Of i-NOS For Use Against Viral Infection

(71) Applicant: UCL Business PLC, London (GB)

(72) Inventors: Christopher O'Callaghan, London (GB); Claire Smith, London (GB); Angrew Easton, London (GB)

(73) Assignee: UCL Business PLC, London (GB)

(21) Appl. No.: 15/754,696

(22) PCT Filed: Jul. 15, 2016
(86) PCT No.: PCT/GB2016/052151

§ 371 Date: Feb. 23, 2018

(30) Foreign Application Priority Data
Jul. 17, 2015 (GB) 1512635.2

(51) Int. Cl. A61K 31/437; A61K 31/155; A61P 31/16; A61P 31/22

(52) U.S. Cl. CPC A61K 31/437; A61P 31/22; A61P 31/16; A61K 31/155

(57) ABSTRACT

The present invention concerns compounds for use in the prevention of viral replication and/or the prevention or treatment of a viral infection, wherein the compounds are selective inhibitors of inducible nitric oxide synthase, and methods of preventing viral replication and/or preventing or treating viral infections in a subject comprising administering a prophylactically or therapeutically effective amount of the compounds.

The present invention relates to uses of therapeutic compounds. In particular, although not exclusively, it concerns inhibitors of nitric oxide synthases and their use in the prevention of viral replication and/or the prevention or treatment of viral infection.

Respiratory viruses are the most frequent cause of hospitalisation of infants and young children in industrialised countries (Shay et al., Bronchiolitis-associated hospitalizations among US children, 1980-1996. JAMA, 1999. 282(15): p. 1440-6.). Respiratory syncytial virus (RSV) is estimated at 64 million cases and 160,000 deaths globally every year, and influenza virus epidemics are estimated to cause 3 to 5 million severe disease cases and 250,000 to 500,000 deaths each year (Stohr, Preventing and treating influenza—Neuraminidase inhibitors are clinically effective but have limitations. British Medical Journal, 2003. 326(7401): p. 1223-1224 and World Health Organisation, Influenza fact sheet 211. 2009). However, strategies to prevent or treat such viral infections are limited.

Most of the antiviral drugs now available are directed at conditions associated with HIV, herpes viruses, the hepatitis B and C viruses, and influenza A and B viruses. However, designing safe and effective antiviral drugs can be difficult, since viruses use the host's cells to replicate. This often makes if challenging to find targets for a drug that would interfere with the virus without also harming the host organism's cells.

In addition, almost all antimicrobials, including antivirals, are subject to drug resistance as the pathogens mutate over time, becoming less susceptible to treatment. Accordingly, there is a constant need for new antiviral drugs.

With this in mind, the inventors have recently discovered that cultured respiratory epithelial cells from patients with primary ciliary dyskinesia (PCD) are more resistant to infection with viruses such as respiratory syncytial virus (RSV) compared to epithelial cells from healthy individuals. This finding therefore provides a new therapeutic strategy against viruses such as RSV.

Patients with PCD exhibit extremely low levels of exhaled and nasal nitric oxide (NO), and it was investigated whether the low viral replication was related to low NO exhibited in these patients. In healthy epithelial cells, NO is produced from L-arginine by three mammalian isoforms of nitric oxide synthase (NOS): neuronal NOS (nNOS), inducible NOS (iNOS, also known as NOS2) and endothelial NOS (eNOS), all of which are expressed within the respiratory tract. Previous studies have suggested a link between low NO and reduced iNOS activity and levels of iNOS and eNOS have both been shown to be very low in children with PCD. This would be consistent with the findings on the clinical effects of selective iNOS inhibitors on exhaled nitric oxide, which show that the majority derives from the iNOS isoform.

Surprisingly, it has now been found that iNOS specific inhibitors strongly inhibit viral replication, which also correlated with a reduction in reactive nitrogen species (RNS) production (particularly NO production) by these cells. In contrast, the non-iNOS specific inhibitor L-NAME was found not to inhibit viral replication. Furthermore, the inventors' findings are in contrast to the study of Stark et al. (J. Infectious Diseases, 2005, 191, 387-395), which has shown that a mildly selective iNOS inhibitor, 2-amino-5,6-dihydro-6-methyl-4H-1,3-thiazine (AMT), actually led to an increase in viral titres in a mouse model of respiratory syncytial virus.

Accordingly, in an aspect of the invention, there is provided a compound for use in the prevention of viral replication and/or the prevention or treatment of a viral infection, wherein the compound is a selective inhibitor of inducible nitric oxide synthase (i.e. an iNOS inhibitor).

The expression ‘selective iNOS inhibitor’ as used herein refers to a chemical entity which demonstrates a distinct selectivity in the inhibition of the iNOS isoform over the other NOS isoforms, i.e. nNOS and eNOS. In particular, a selective iNOS inhibitor may be defined as possessing a ≥30 fold selectivity for iNOS over the other NOS isoforms, preferably a ≥40 or ≥45 fold selectivity for iNOS, or more preferably a ≥50 fold selectivity for iNOS, under comparable assay conditions.

The inhibition of viral replication has been observed with a number of structurally diverse iNOS inhibitors. Thus, based on the data that has been generated, the prevention of viral replication is linked to a reduction in iNOS activity and, as such, is applicable to any known compound which is a selective inhibitor of inducible nitric oxide synthase.

In a more specific embodiment, there is provided a compound for use according to the invention, wherein the compound is of the formula:

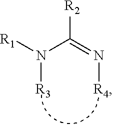

wherein
   R₁ is hydrogen, an optionally substituted C₁₋₆ alkyl group, an optionally substituted C₂₋₆ alkenyl group, an optionally substituted C₆₋₁₀ aryl group, an optionally substituted C₇₋₁₆ aralkyl group, an optionally substituted 5- to 10-membered heterocyclyl group, or a group of the formula:

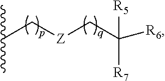

wherein
   Z is an optionally substituted C₆₋₁₀ arylene group, an optionally substituted C₁₋₆ alkylene group, an optionally substituted C₂₋₆ alkenylene group, an optionally substituted 5- to 10-membered heterocyclylene group, a group of the formula —S(O)ₓ—, where x is 0, 1, or 2, a group of the formula —NR₈—, where R₈ is hydrogen, a C₁₋₆ alkyl group, or a C₆₋₁₀ aryl group, or a group of the formula —O—;
   p is an integer from 0 to 5;
   q is an integer from 0 to 5;
   R₅ is hydrogen, an optionally substituted C₁₋₆ alkyl group, or an optionally substituted C₁₋₆ alkoxy group;
   R₆ is a carboxyl group, an optionally substituted C₁₋₆ alkyl carbonyloxy group, an optionally substituted C₁₋₆ alkyl carbonyl group, an optionally substituted C₁₋₆ alkoxy carbonyl group, a carbamoyl group, or an optionally substituted C₁₋₆ alkyl carbamoyl group; and
   R₇ is an optionally mono- or di-substituted amino group or an optionally substituted C₁₋₆ alkoxy group;
R₂ is hydrogen, an optionally substituted C₁₋₆ alkyl group, an optionally substituted C₂₋₆ alkenyl group, an optionally substituted C₂₋₆ alkynyl group, an optionally substituted C₃₋₆ cycloalkyl group, an optionally substituted C₃₋₆ cycloalkyl-C₁₋₆ alkyl group, an optionally substituted C₇₋₁₆ aralkyl group, or an optionally substituted C₆₋₁₀ aryl group;
R₃ is hydrogen, an optionally substituted C₁₋₆ alkyl group, or an optionally substituted C₆₋₁₀ aryl group; and
R₄ is hydrogen, an optionally substituted C₁₋₆ alkyl group, or an optionally substituted C₆₋₁₀ aryl group;
or R₃ and R₄ are joined together to form an optionally substituted 5- to 10-membered monocyclic or bicyclic heterocyclyl group;
provided that R₁, R₂, R₃, and R₄ are not all hydrogen;
or a pharmaceutically acceptable salt thereof.

The term ‘C_{x-y} alkyl’ as used herein refers to a linear or branched saturated hydrocarbon group containing from x to y carbon atoms. Examples of C₁₋₆ alkyl groups include methyl, ethyl, n-propyl, isopropyl, n-butyl, isobutyl, sec-butyl, tert-butyl, n-pentyl, isopentyl, neopentyl, hexyl, and isohexyl.

The term ‘C_{x-y} alkylene’ as used herein refers to a divalent hydrocarbon group obtained by removing one hydrogen atom from ‘C_{x-y} alkyl’ above. Examples of C₁₋₆ alkylene groups include methylene, ethylene, propylene, butylene, pentylene, and hexylene.

The term ‘C_{x-y} alkenyl’ as used herein refers to a linear or branched hydrocarbon group containing one or more carbon-carbon double bonds and having from x to y carbon atoms. Examples of C₂₋₆ alkenyl groups include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, and 5-hexenyl. The term ‘C_{x-y} alkenylene’ as used herein refers to a divalent hydrocarbon group obtained by removing one hydrogen atom from ‘C_{x-y} alkenyl’ above. Examples of C₂₋₆ alkenylene groups include ethenylene, propenylene, butenylene, 1,3-butadienylene, pentenylene, hexenylene, and 1,3,5-hexatrienylene.

The term ‘C_{x-y} alkynyl’ as used herein refers to a divalent hydrocarbon group containing one or more carbon-carbon triple bonds and having from x to y carbon atoms. Examples of C₂₋₆ alkynyl groups include ethynyl, propynyl, butynyl and pentynyl.

The term ‘C_{x-y} alkoxy’ as used herein refers to an —O—C_{x-y} alkyl group wherein C_{x-y} alkyl is as defined herein. Examples of C₁₋₆ alkoxy groups include methoxy, ethoxy, propoxy, iso-propoxy, butoxy, tert-butoxy, pentoxy and hexoxy.

The term ‘C_{x-y} cycloalkyl’ as used herein refers to a saturated monocyclic hydrocarbon ring of x to y carbon atoms. Examples of C₃₋₆ cycloalkyl groups include cyclopropyl, cyclobutyl, cyclopentyl, and cyclohexyl.

The term ‘C_{x-y} aryl’ as used herein refers to a monocyclic or bicyclic ring containing from x to y carbon atoms, wherein at least one ring is aromatic. Examples of C₆₋₁₀ aryl groups include phenyl, naphthyl, tetrahydronaphthalenyl, anthryl, phenanthryl, acenaphthylenyl, and biphenylyl.

The term ‘C_{x-y} arylene’ as used herein refers to a divalent hydrocarbon group obtained by removing one hydrogen atom from ‘C_{x-y} aryl’ above. Examples of C₆₋₁₀ arylene groups include phenylene, naphthylene, tetrahydronaphthalenylene, anthrylene, phenanthrylene, acenaphthylenylene, and biphenylylene.

The term ‘C_{x-y} aralkyl’ as used herein refers to a linear or branched saturated hydrocarbon group linked to an aryl group containing from x to y carbon atoms in total. Examples of C₇₋₁₆ aralkyl groups include benzyl, phenethyl, naphthylmethyl, and biphenylylmethyl. C₇₋₁₂ aralkyl groups are preferred.

The term ‘x- to y-membered heterocyclyl’ refers to a monocyclic or bicyclic ring which may be saturated or partially unsaturated (i.e. non-aromatic), or fully unsaturated (i.e. aromatic), in which the monocyclic or bicyclic ring contains x to y ring atoms, of which 1 to 4 are heteroatoms selected from oxygen, nitrogen, and sulphur. Examples of such non-aromatic monocyclic rings include aziridinyl, oxiranyl, pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl and azepanyl. Examples of such bicyclic non-aromatic rings include tetrahydroquinolinyl, tetrahydroisoquinolinyl, dihydroquinolinyl, dihydroisoquinolinyl, dihydroindolyl, dihydroisoindolyl, dihydrobenzofuryl, dihydroisobenzofuryl, tetrahydroquinazolinyl, dihydroquinazolinyl, dihydrobenzoxazolyl, and dihydrobenzimidazolyl. Examples of such monocyclic aromatic rings include thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, and tetrazinyl. Examples of such bicyclic aromatic rings include quinolinyl, isoquinolinyl, indolyl, isoindolyl, benzofuryl, isobenzofuryl, quinazolinyl, benzoxazolyl, and benzimidazole.

In cases where the heterocyclyl group is divalent, i.e. is a ‘heterocyclylene’ group, the group may be obtained by removing one hydrogen atom from the ‘heterocyclyl’ group above.

The term ‘haloC_{x-y} alkyl’ as used herein refers to a C_{x-y} alkyl group as defined herein wherein at least one hydrogen atom is replaced with halogen. Examples of haloC₁₋₆ alkyl groups include fluoroethyl, trifluoromethyl and trifluoroethyl.

The term ‘C_{x-y} cycloalkyl-C_{x-y} alkyl’ as used herein refers C_{x-y} cycloalkyl group as defined herein joined via a C_{x-y} alkyl group as defined herein. Examples of C₃₋₆ cycloalkyl-C₁₋₆ alkyl groups include methyl cyclopropyl, methyl cyclobutyl, methyl cyclopentyl, methyl cyclohexyl, and ethyl, butyl, pentyl, and hexyl variants thereof.

The term ‘amino’ as used herein refers to an organonitrogen compound with the connectivity —N(R′)(R″), where R′ and R″ are each independently hydrogen or a group selected from C₁₋₆ alkyl, tert-butoxycarbonyl, benzyl, a group of the formula —COR′″, wherein R′″ is hydrogen or a C₁₋₆ alkyl group, and a group of the formula —S(O)_{m′}R″″, wherein m′ is 0, 1 or 2 and R″″ is hydrogen or a C₁₋₆ alkyl group.

The term ‘C_{x-y} alkoxy carbonyl’ as used herein refers to an alkyl group wherein C_{x-y} alkyl is as defined herein and at least one methylene group (i.e. —CH₂—) is replaced with an ester group (e.g. —OC(O)—). Examples of C₁₋₆ alkyl carbonyl groups include ethyl oxycarbonyl, propyl oxycarbonyl, butyl oxycarbonyl, pentyl oxycarbonyl, and hexyl oxycarbonyl. The term ‘oxycarbonyl’ as used herein refers to a single oxycarbonyl group of the formula: —OC(O)—. The term ‘carboxyl’ as used herein refers to a single carboxyl group of the formula: —CO₂H.

The term ‘C_{x-y} alkyl carbonyloxy’ as used herein refers to an alkyl group wherein C_{x-y} alkyl is as defined herein and at least one methylene group (i.e. —CH₂—) is replaced with an ester group (e.g. —CO₂—). Examples of C₁₋₆ alkyl carbonyloxy groups include ethanoate, propanoate, butanoate, pentanoate, and hexanoate. The term ‘carbonyloxy’ as used herein refers to a single carbonyloxy group of the formula: —CO₂—.

The term ‘C_{x-y} alkyl carbamoyl’ as used herein refers to an alkyl group wherein C_{x-y} alkyl is as defined herein and at least one methylene group (i.e. —CH₂—) is replaced with an amide group (e.g. —C(O)NR—, where R is a hydrogen atom, a 5- or 6-membered heterocyclyl group, a C₃₋₆ cycloalkyl group, a C₁₋₆ alkyl group, or a C₆₋₁₄ aryl group, preferably a hydrogen atom). Examples of C₁₋₆ alkyl carbamoyl groups include ethyl carbamoyl, propyl carbamoyl, butyl carbamoyl, pentyl carbamoyl, and hexyl carbamoyl. The term ‘carbamoyl’ as used herein refers to a single carbamoyl group of the formula: —C(O)NH₂.

The term ‘halogen’ as used herein refers to a fluorine, chlorine, bromine or iodine atom, and any radioactive isotope thereof, including fluorine-18, iodine-123, iodine-124, iodine-125, and iodine-131, unless otherwise specified.

Where a given structural group is described herein as ‘optionally substituted’, the optional substituents of that group are as follows:

(1) 1 to 3 groups (preferably 1 or 2 groups) selected from -J-C₆₋₁₀ aryl, -J-5-or-6-membered heterocyclyl and -J-C₃₋₆ cycloalkyl, wherein J represents a bond, O or C₁₋₆ alkylene, and said 5-or-6-membered heterocyclyl is selected from triazolyl, thiazolyl, thienyl, pyrazolyl, pyrimidyl, pyridazinyl, pyrazinyl, pyridyl, pyrrolidinyl, azetidinyl, pyrazolidinyl, oxazolidinyl, piperidinyl, piperazinyl, morpholinyl, thiomorpholinyl, and thiazolidinyl, and said C₃₋₆ cycloalkyl is selected from cyclopropyl, cyclobutyl, cyclopentyl and cyclohexyl, and wherein the C₆₋₁₀ aryl groups is optionally substituted by 1 to 3 substituents selected from a halogen atom and a group of the formula —SO₂R′, wherein R′ is hydrogen, a C₁₋₃ alkyl group, a group of the formula —NR″R′″, wherein R″ and R′″ are each individually selected from hydrogen and a C₁₋₃ alkyl group, or both are joined together to form a 6-membered heterocyclyl group optionally substituted by a C₁₋₃ alkyl group, and the 5-or-6-membered heterocyclyl and C₃₋₆ cycloalkyl groups are each independently optionally substituted by 1 to 3 substituents selected from halogen, C₁₋₆ alkyl, C₁₋₆ alkoxy, and haloC₁₋₆ alkyl; and/or

(2) 1 to 3 substituents selected from

(a) C₁₋₆ alkyl (preferably methyl, ethyl or isopropyl),

(b) C₁₋₆ alkenyl (preferably propenyl),

(c) C₁₋₆ alkynyl (preferably ethynyl or propynyl),

(d) halogen (preferably bromo, chloro or fluoro),

(e) haloC₁-6 alkyl (preferably trifluoromethyl),

(f) —CN,

(g) amino, optionally mono- or di-substituted with C₁₋₆ alkyl, tert-butoxycarbonyl, benzyl, a group of the formula —COR′″, wherein R′″ is hydrogen or a C₁₋₆ alkyl group, or a group of the formula —S(O)_{m′}R″″, wherein m′ is 0, 1 or 2 and R″″ is hydrogen or a C₁₋₆ alkyl group,

(h) C₁₋₆ alkoxy (preferably methoxy) optionally substituted by 1 to 3 halogen atoms, a C₆₋₁₀ aryl group or a group of the formula —COR²¹, wherein R²¹ is hydrogen or a C₁₋₆ alkyl group,

(i) C₁₋₆ alkyl carbonyl, including ketones and derivatives thereof such as ketals and hemiketals, and aldehydes (e.g. formyl) and derivatives thereof such as acetals and hemiacetals (preferably acetyl),

(j) C₁₋₆ alkoxy carbonyl,

(k) C₁₋₆ alkyl carbonyloxy, including carboxyl,

(l) C₁₋₆ alkyl carbamoyl, including carbamoyl,

(m) —NO₂,

(n) —OH,

(o) —SH,

(p) —S(O)ₘR′, wherein m is 0, 1 or 2, R′ is hydrogen, a C₁₋₆ alkyl group, hydroxy or a group of the formula NR″R′″, wherein R″ and R′″ are independently hydrogen or a C₁₋₆ alkyl group, and

(q) —PO(OR′)₂, wherein R′ is hydrogen or a C₁₋₆ alkyl group.

In a preferred embodiment, R₁ is a group of the formula:

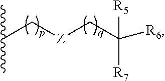

wherein
   Z is (i) a C₆₋₁₀ arylene group (preferably phenyl) optionally substituted by 1 to 3 substituents independently selected from halogen, a C₁₋₆ alkyl group, and a C₁₋₆ alkoxy group, (ii) a C₂₋₆ alkenylene group optionally substituted by 1 to 3 substituents independently selected from halogen and a C₁₋₆ alkyl group, (iii) a group of the formula —S(O)ₓ—, where x is 0, 1, or 2, (iv) a group of the formula —O—, or (v) a C₁₋₃ alkylene group;
   p is 0, 1, or 2;
   q is 0, 1, or 2;
   R₅ is hydrogen or a C₁₋₆ alkyl group;
   R₆ is a carboxyl group, a C₁₋₆ alkoxy carbonyl group, a carbamoyl group optionally substituted by a 5- or 6-membered heterocyclyl group, or a C₁₋₆ alkyl carbamoyl group; and
   R₇ is an amino group optionally mono- or di-substituted by a C₁₋₆ alkyl group or a C₇₋₁₀ aralkyl group.

For example, specifically preferred compounds of this embodiment are described in Table 1 below, including pharmaceutically acceptable salts thereof.

TABLE 1

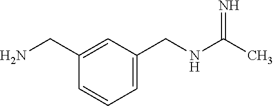

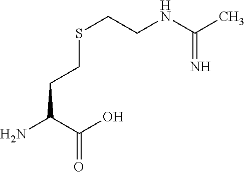

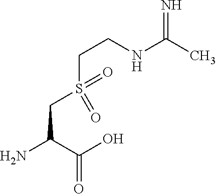

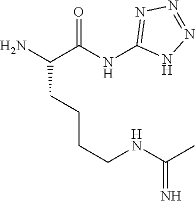

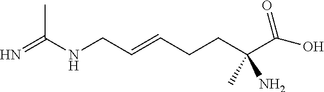

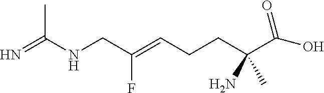 [SMILES: CC(=N)NC/C(F)=C/CC[C@](C)(N)C(=O)O]

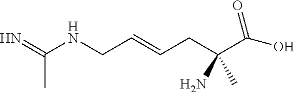

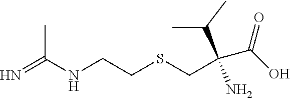

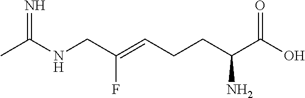

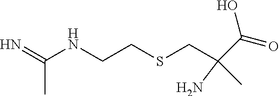

Preferably, R₂ is hydrogen, or a C₁₋₆ alkyl group, a C₂₋₆ alkenyl group, a C₂₋₆ alkynyl group, or a C₃₋₆ cycloalkyl-C₁₋₆ alkyl group, each of which is optionally substituted by 1 to 3 substituents independently selected from:
(i) —CN;
(ii) —NO₂;
(iii) a group of the formula —COR⁸, wherein R⁸ is hydrogen, a C₁₋₆ alkyl group, a group of the formula —OR⁹, wherein R⁹ is hydrogen or C₁₋₆ alkyl, or a group of the formula NR¹⁰R¹¹, wherein R¹⁰ and R¹¹ are independently selected from hydrogen or a C₁₋₆ alkyl group;
(iv) a group of the formula —S(O)ₘR¹², wherein m is 0, 1 or 2, R¹² is hydrogen, a C₁₋₆ alkyl group, hydroxy or a group of the formula NR¹³R¹⁴, wherein R¹³ and R¹⁴ are independently hydrogen or a C₁₋₆ alkyl group;
(v) a group of the formula PO(OR¹⁵)₂, wherein R¹⁵ is hydrogen or a C₁₋₆ alkyl group;
(vi) a group of the formula NR¹⁶R¹⁷, wherein R¹⁶ and R¹⁷ are independently selected from hydrogen, a C₁₋₆ alkyl group, a group of the formula —COR¹⁸, wherein R¹⁸ is hydrogen or a C₁₋₆ alkyl group, or a group of the formula —S(O)_{m′}R¹⁹, wherein m′ is 0, 1 or 2 and R¹⁹ is hydrogen or a C₁₋₆ alkyl group;
(vii) a halogen atom; and
(viii) a group of the formula —OR²⁰, wherein R²⁰ is hydrogen, a C₁₋₆ alkyl group optionally substituted by 1 to 3 halogen atoms, a C₆₋₁₀ aryl group or a group of the formula —COR²¹, wherein R²¹ is hydrogen or a C₁₋₆ alkyl group.

In another preferred embodiment, R₂ is hydrogen, or a C₁₋₆ alkyl group optionally substituted by a C₆₋₁₀ aryl group or a 5- to 7-membered heterocyclyl group, each of which is optionally substituted by 1 to 3 substituents independently selected from:
(i) —NH₂;
(ii) —OH;
(iii) a halogen atom;
(iv) a C₁₋₆ alkyl group; and
(v) a C₁₋₆ alkoxy group.

In yet another preferred embodiment, R₃ is hydrogen, or a C₁₋₆ alkyl group, and R₄ is hydrogen, or a C₁₋₆ alkyl group.

In an alternative preferred embodiment, R₃ and R₄ are joined together to form a 5- or 6-membered monocyclic heterocyclyl group or a 9- or 10-membered bicyclic heterocyclyl group, each of which is optionally substituted by 1 to 3 substituents independently selected from:
(i) —NH₂;
(ii) —OH;
(iii) a halogen atom;
(iv) a C₁₋₆ alkyl group;
(v) a C₁₋₆ alkoxy group;
(vi) a C₆₋₁₀ aryl group optionally substituted by 1 to 3 substituents independently selected from: a halogen atom and a group of the formula —SO₂R²², wherein R²² is hydrogen, a C₁₋₃ alkyl group, a group of the formula —NR²³R²⁴, wherein R²³ and R²⁴ are each individually selected from hydrogen and a C₁₋₃ alkyl group, or both are joined together to form a 6-membered heterocyclyl group optionally substituted by a C₁₋₃ alkyl group.

For example, specifically preferred compounds of this embodiment are described in Table 2 below, including pharmaceutically acceptable salts thereof.

TABLE 2

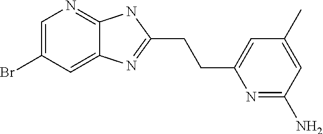 [SMILES: Cc1cc(N)nc(CCc2nc3cc(Br)cnc3[nH]2)c1]

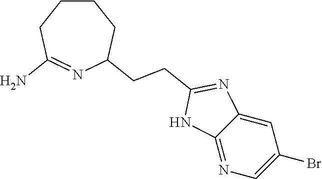 [SMILES: NC1=NC(CCc2nc3cc(Br)cnc3[nH]2)CCCC1]

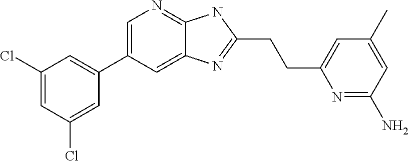 [SMILES: Cc1cc(N)nc(CCc2nc3cc(-c4cc(Cl)cc(Cl)c4)cnc3[nH]2)c1]

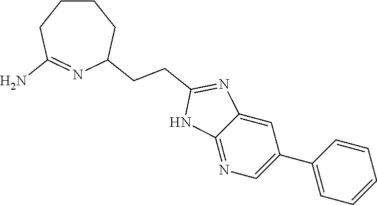 [SMILES: NC1=NC(CCc2nc3cc(-c4ccccc4)cnc3[nH]2)CCCC1]

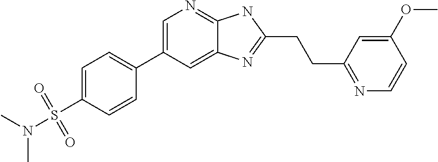

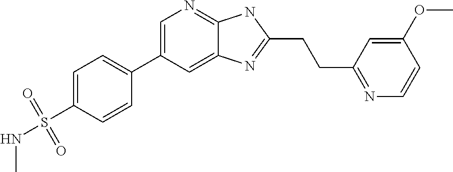 [SMILES: CNS(=O)(=O)c1ccc(-c2cnc3[nH]c(CCc4cc(OC)ccn4)nc3c2)cc1]

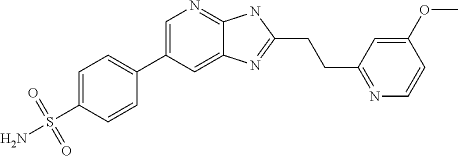 [SMILES: COc1ccnc(CCc2nc3cc(-c4ccc(S(N)(=O)=O)cc4)cnc3[nH]2)c1]

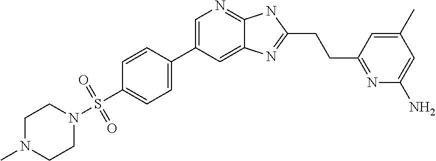 [SMILES: Cc1cc(N)nc(CCc2nc3cc(-c4ccc(S(=O)(=O)N5CCN(C)CC5)cc4)cnc3[nH]2)c1]

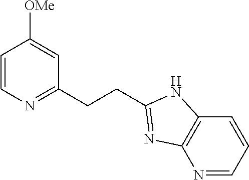 [SMILES: COc1ccnc(CCc2nc3ncccc3[nH]2)c1]

In a particularly preferred embodiment, the compound for use according to the invention is a compound of the formula:

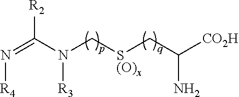

wherein
   R₂ is a C₁₋₆ alkyl group, a C₂₋₆ alkenyl group, a C₂₋₆ alkynyl group, or a C₃₋₆ cycloalkyl-C₁₋₆ alkyl group, each of which is optionally substituted by 1 to 3 substituents independently selected from:
      (i) —CN;
      (ii) —NO₂;
      (iii) a group of the formula —COR⁸, wherein R⁸ is hydrogen, a C₁₋₆ alkyl group, a group of the formula —OR⁹, wherein R⁹ is hydrogen or C₁₋₆ alkyl, or a group of the formula NR¹⁰R¹¹, wherein R¹⁰ and R¹¹ are independently selected from hydrogen or a C₁₋₆ alkyl group;
      (iv) a group of the formula —S(O)ₘR¹², wherein m is 0, 1 or 2, R¹² is hydrogen, a C₁₋₆ alkyl group, hydroxy or a group of the formula NR¹³R¹⁴ wherein R¹³ and R¹⁴ are independently hydrogen or a C₁₋₆ alkyl group;
      (v) a group of the formula PO(OR¹⁵)₂, wherein R¹⁵ is hydrogen or a C₁₋₆ alkyl group;
      (vi) a group of the formula NR¹⁶R¹⁷, wherein R¹⁶ and R¹⁷ are independently selected from hydrogen, a C₁₋₆ alkyl group, a group of the formula —COR¹⁸, wherein R¹⁸ is hydrogen or a C₁₋₆ alkyl group, or a group of the formula —S(O)_{m′}R¹⁹, wherein m′ is 0, 1 or 2 and R¹⁹ is hydrogen or a C₁₋₆ alkyl group;
      (vii) a halogen atom; and
      (viii) a group of the formula —OR²⁰, wherein R²⁰ is hydrogen, a C₁₋₆ alkyl group optionally substituted by 1 to 3 halogen atoms, a C₆₋₁₀ aryl group or a group of the formula —COR²¹, wherein R²¹ is hydrogen or a C₁₋₆ alkyl group;
   R₃ and R₄ are each hydrogen;
   p is 2 or 3;
   q is 1 or 2; and
   x is 1 or 2.

Preferably, in this embodiment, R₂ is a C₁₋₄ alkyl group, a C₂₋₄ alkenyl group, or a C₂₋₄ alkynyl group, each of which is optionally substituted by 1 to 3 substituents independently selected from: —CN; halogen; a group of the formula —COR⁸, wherein R⁸ is hydrogen, a C₁₋₆ alkyl group, a group of the formula —OR⁹, wherein R⁹ is hydrogen or C₁₋₆ alkyl, or a group of the formula NR¹⁰R¹¹, wherein R¹⁰ and R¹¹ are independently selected from hydrogen or a C₁₋₆ alkyl group; a group of the formula —S(O)ₘR¹², wherein m is 0, 1 or 2, R¹² is hydrogen, a C₁₋₆ alkyl group, hydroxy or a group of the formula NR¹³R¹⁴, wherein R¹³ and R¹⁴ are independently hydrogen or a C₁₋₆ alkyl group; a group of the formula PO(OR¹⁵)₂, wherein R¹⁵ is hydrogen or a C₁₋₆ alkyl group; a group of the formula NR¹⁶R¹⁷, wherein R¹⁶ and R¹⁷ are independently selected from hydrogen, a C₁₋₆ alkyl group, a group of the formula —COR¹⁸, wherein R¹⁸ is hydrogen or a C₁₋₆ alkyl group, or a group of the formula —S(O)_{m′}R¹⁹, wherein m′ is 0, 1 or 2 and R¹⁹ is hydrogen or a C₁₋₆ alkyl group; and a group of the formula —OR²⁰, wherein R²⁰ is hydrogen, a C₁₋₆ alkyl group optionally substituted by 1 to 3 halogen atoms, a C₆₋₁₀ aryl group or a group of the formula —COR²¹, wherein R²¹ is hydrogen or a C₁₋₆ alkyl group.

More preferably, R₂ is a methyl or ethyl group, each of which is optionally substituted by 1 to 3 substituents independently selected from: halogen; a group of the formula —S(O)ₘR¹², wherein m is 0, 1 or 2, R¹² is hydrogen, a C₁₋₆ alkyl group, hydroxy or a group of the formula NR¹³R¹⁴, wherein R¹³ and R¹⁴ are independently hydrogen or a C₁₋₆ alkyl group; (v) a group of the formula PO(OR¹⁵)₂, wherein R¹⁵ is hydrogen or a C₁₋₆ alkyl group; and a group of the formula —OR²⁰, wherein R²⁰ is hydrogen, a C₁₋₆ alkyl group optionally substituted by 1 to 3 halogen atoms, a C₆₋₁₀ aryl group or a group of the formula —COR²¹, wherein R²¹ is hydrogen or a C₁₋₆ alkyl group. Most preferably, R₂ is methyl.

In particular, the compound for use according to the invention is preferably a compound selected from:

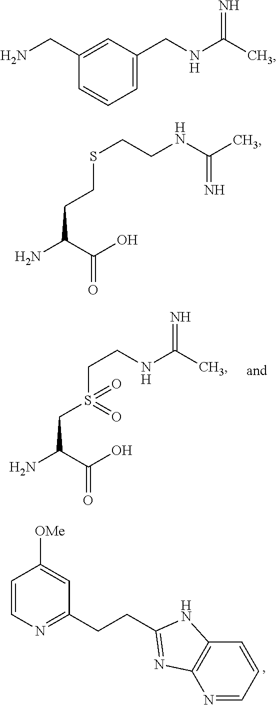

and pharmaceutically acceptable salts thereof.

In an alternative embodiment, the selective iNOS inhibitor for use in the invention may be a compound according to any of the following formulae, as described in the following identified patent or literature references. Specifically, a compound according to:
Formula (I) of WO 95/34534;
Formula (I) of WO 2005/030768;
Formula (I) of WO 2005/030770;
Formula (I) of WO 2007/039578;
Formula (I) of WO 2007/045622;
Formula (I) of WO 2005/030769;
Formula (I) of WO 2005/030771;
Formula (I) of WO 03/080607;
Formula (I) of WO 2005/061496;
Formula (I) of WO 2005/026143;
Formula (I) of WO 2008/031788;
Formula (I) of WO 2006/103255;
Formula (I), (II) or (III) of WO 03/092678;
Formula (Ya), (Yb), or (Yc) of WO 01/14371;
Formula (I) of US 2003/0069210;
Formula 1 of WO 2008/072937;
Formula 1 of WO 01/72703;
Formula 1 of WO 01/72702;
Formula I of WO 02/22559;
Formula I of WO 2005/025620;
Formula I of WO 02/22562;
Formula (I) of WO 91/13055;
Formula (I) of WO 95/25717;
Formula (II) of WO 2006/060424;
Formula (I) of WO 2007/062410;
Formula (I) of WO 2007/062417;
Formula (I) of WO 2007/117778;
Formula (I) of WO 2008/103615;
Formula I or II of WO 2009/029625
Formula I of WO 2007/084868;
Formula I or VI of WO 2007/101213;
Formula (I) of WO 2004/041794;
Formula (I) of WO 2004/009580;
Formula (I) of WO 03/011831;
Formula (I) of WO 03/029185;
Formula (I) of WO 2004/009579,
Formula (III) of CN 102702298,
Formula 2 of Schulz et al., Bioorg. Med. Chem., 2013, 21 (17), 5518-5531,
Formula (II) of WO 2006/060424,
Formula (II) of WO 2009/029617,
Formula (I) of WO 2005/030768,
Compounds 1 and 2 of Lee at al., J. Nat. Prod. 2014, 77 (6), 1528-1531,
Compounds 3a-g of Stefani et al., Euro. J. Med. Chem. 2012, 58, 117-127, or
Compounds 1-158 of Suaifan et al., J. Mol. Graph. Model. 2012, 37, 1-26, including pharmaceutically acceptable salts thereof.

More specifically, the compound for use according to the invention may be one of the compounds presented below in Table 3, each of which is a known selective inhibitor of iNOS, including pharmaceutically acceptable salts thereof.

TABLE 3

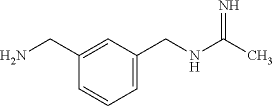

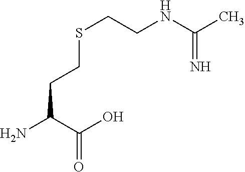

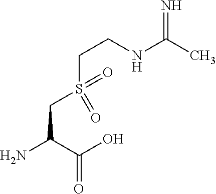

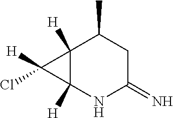

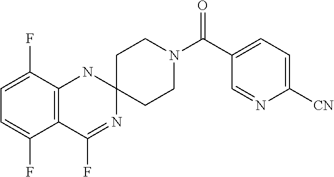 [SMILES: N#Cc1ccc(C(=O)N2CCC3(CC2)N=C(F)c2c(F)ccc(F)c2N3)cn1]

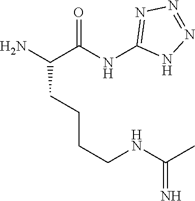

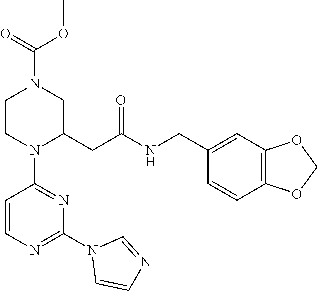 [SMILES: COC(=O)N1CCN(c2ccnc(-n3ccnc3)n2)C(CC(=O)NCc2ccc3c(c2)OCO3)C1]

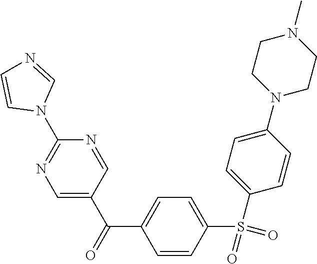 [SMILES: CN1CCN(c2ccc(S(=O)(=O)c3ccc(C(=O)c4cnc(-n5ccnc5)nc4)cc3)cc2)CC1]

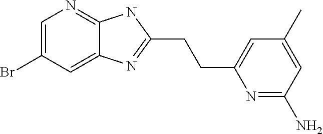 [SMILES: Cc1cc(N)nc(CCc2nc3cc(Br)cnc3[nH]2)c1]

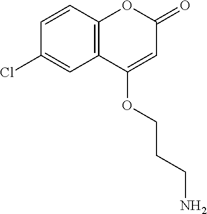 [SMILES: NCCCOc1cc(=O)oc2ccc(Cl)cc12]

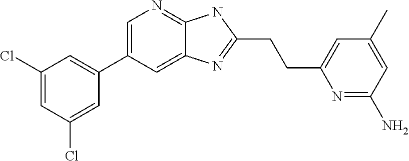 [SMILES: Cc1cc(N)nc(CCc2nc3cc(-c4cc(Cl)cc(Cl)c4)cnc3[nH]2)c1]

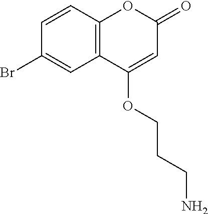 [SMILES: NCCCOc1cc(=O)oc2ccc(Br)cc12]

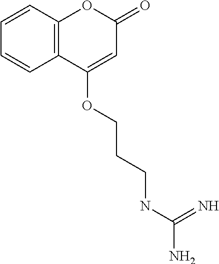 [SMILES: N=C(N)NCCCOc1cc(=O)oc2ccccc12]

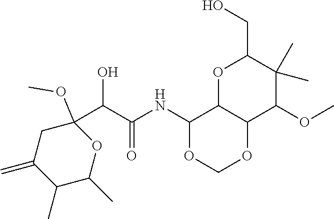

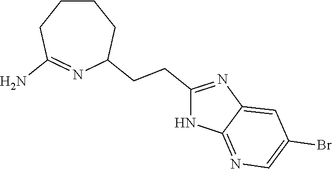 [SMILES: NC1=NC(CCc2nc3cc(Br)cnc3[nH]2)CCCC1]

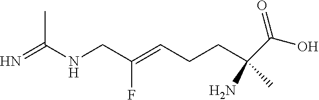 [SMILES: CC(=N)NC/C(F)=C/CC[C@](C)(N)C(=O)O]

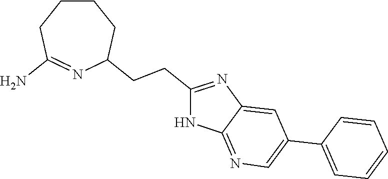 [SMILES: NC1=NC(CCc2nc3cc(-c4ccccc4)cnc3[nH]2)CCCC1]

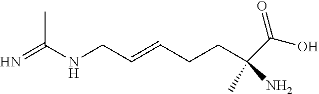

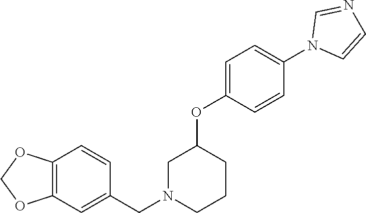 [SMILES: c1cn(-c2ccc(OC3CCCN(Cc4ccc5c(c4)OCO5)C3)cc2)cn1]

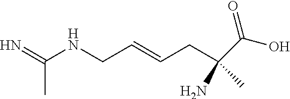

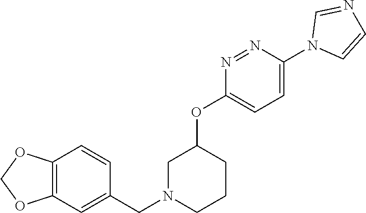 [SMILES: c1cn(-c2ccc(OC3CCCN(Cc4ccc5c(c4)OCO5)C3)nn2)cn1]

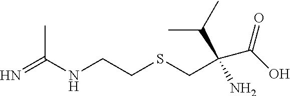

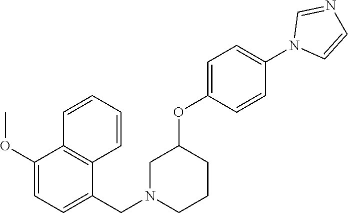 [SMILES: COc1ccc(CN2CCCC(Oc3ccc(-n4ccnc4)cc3)C2)c2ccccc12]

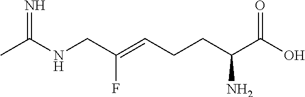

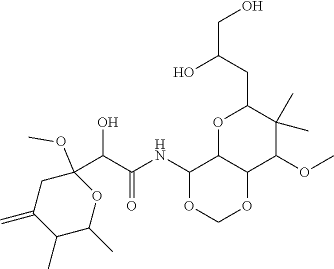

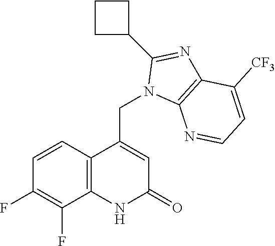 [SMILES: O=c1cc(Cn2c(C3CCC3)nc3c(C(F)(F)F)ccnc32)c2ccc(F)c(F)c2[nH]1]

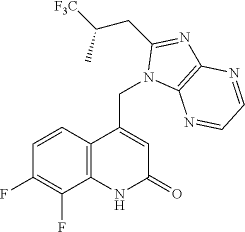 [SMILES: C[C@@H](Cc1nc2nccnc2n1Cc1cc(=O)[nH]c2c(F)c(F)ccc12)C(F)(F)F]

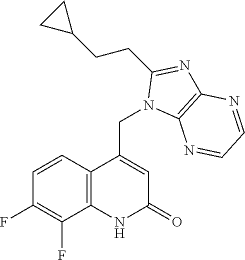 [SMILES: O=c1cc(Cn2c(CCC3CC3)nc3nccnc32)c2ccc(F)c(F)c2[nH]1]

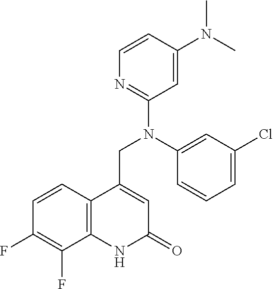 [SMILES: CN(C)c1ccnc(N(Cc2cc(=O)[nH]c3c(F)c(F)ccc23)c2cccc(Cl)c2)c1]

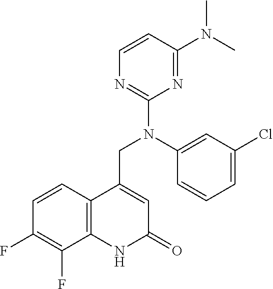 [SMILES: CN(C)c1ccnc(N(Cc2cc(=O)[nH]c3c(F)c(F)ccc23)c2cccc(Cl)c2)n1]

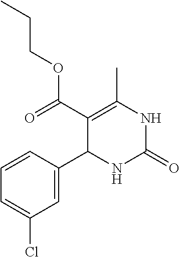 [SMILES: CCCOC(=O)C1=C(C)NC(=O)NC1c1cccc(Cl)c1]

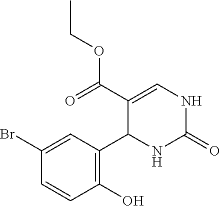 [SMILES: CCOC(=O)C1=CNC(=O)NC1c1cc(Br)ccc1O]

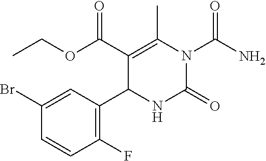 [SMILES: CCOC(=O)C1=C(C)N(C(N)=O)C(=O)NC1c1cc(Br)ccc1F]

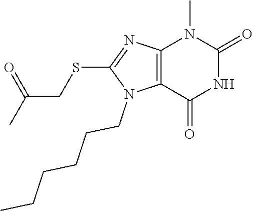 [SMILES: CCCCCCn1c(SCC(C)=O)nc2c1c(=O)[nH]c(=O)n2C]

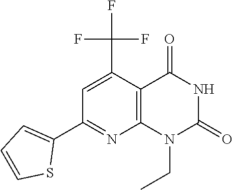 [SMILES: CCn1c(=O)[nH]c(=O)c2c(C(F)(F)F)cc(-c3cccs3)nc21]

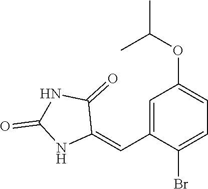 [SMILES: CC(C)Oc1ccc(Br)c(/C=C2/NC(=O)NC2=O)c1]

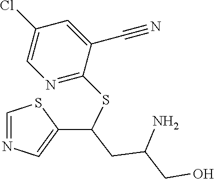 [SMILES: N#Cc1cc(Cl)cnc1SC(CC(N)CO)c1cncs1]

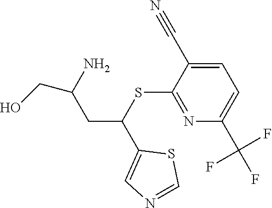 [SMILES: N#Cc1ccc(C(F)(F)F)nc1SC(CC(N)CO)c1cncs1]

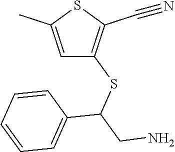 [SMILES: Cc1cc(SC(CN)c2ccccc2)c(C#N)s1]

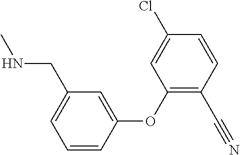 [SMILES: CNCc1cccc(Oc2cc(Cl)ccc2C#N)c1]

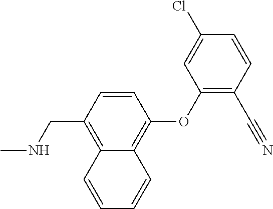 [SMILES: CNCc1ccc(Oc2cc(Cl)ccc2C#N)c2ccccc12]

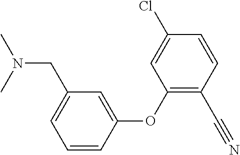 [SMILES: CN(C)Cc1cccc(Oc2cc(Cl)ccc2C#N)c1]

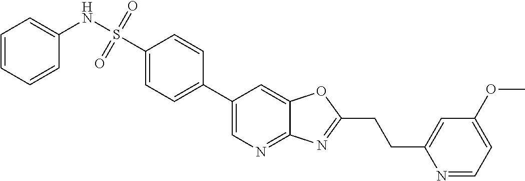 [SMILES: COc1ccnc(CCc2nc3ncc(-c4ccc(S(=O)(=O)Nc5ccccc5)cc4)cc3o2)c1]

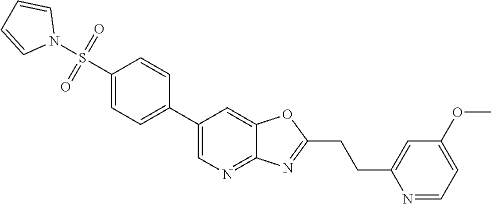 [SMILES: COc1ccnc(CCc2nc3ncc(-c4ccc(S(=O)(=O)n5cccc5)cc4)cc3o2)c1]

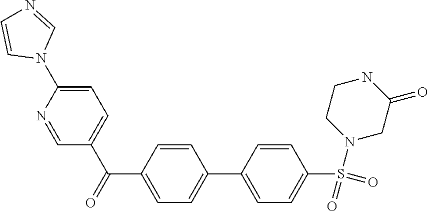 [SMILES: O=C1CN(S(=O)(=O)c2ccc(-c3ccc(C(=O)c4ccc(-n5ccnc5)nc4)cc3)cc2)CCN1]

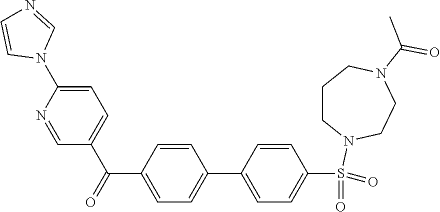 [SMILES: CC(=O)N1CCCN(S(=O)(=O)c2ccc(-c3ccc(C(=O)c4ccc(-n5ccnc5)nc4)cc3)cc2)CC1]

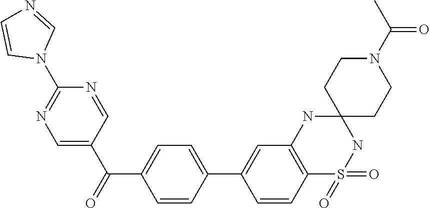 [SMILES: CC(=O)N1CCC2(CC1)Nc1cc(-c3ccc(C(=O)c4cnc(-n5ccnc5)nc4)cc3)ccc1S(=O)(=O)N2]

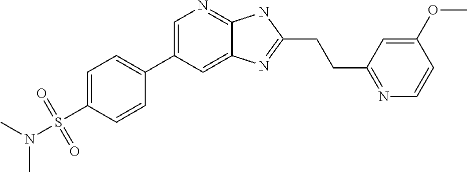

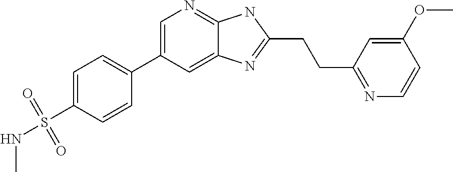 [SMILES: CNS(=O)(=O)c1ccc(-c2cnc3[nH]c(CCc4cc(OC)ccn4)nc3c2)cc1]

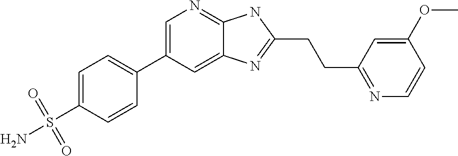 [SMILES: COc1ccnc(CCc2nc3cc(-c4ccc(S(N)(=O)=O)cc4)cnc3[nH]2)c1]

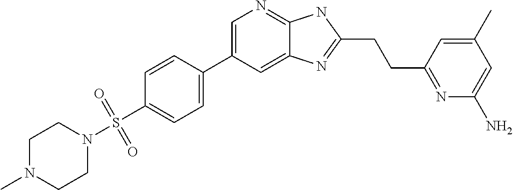 [SMILES: Cc1cc(N)nc(CCc2nc3cc(-c4ccc(S(=O)(=O)N5CCN(C)CC5)cc4)cnc3[nH]2)c1]

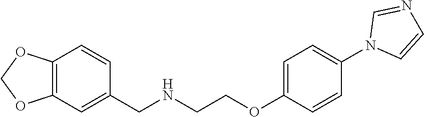 [SMILES: c1cn(-c2ccc(OCCNCc3ccc4c(c3)OCO4)cc2)cn1]

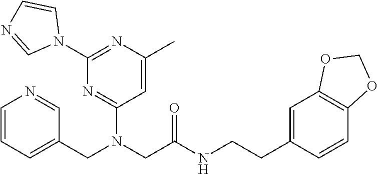 [SMILES: Cc1cc(N(CC(=O)NCCc2ccc3c(c2)OCO3)Cc2cccnc2)nc(-n2ccnc2)n1]

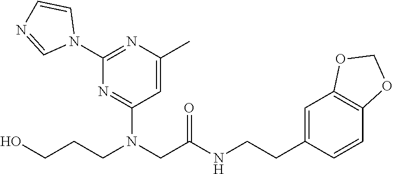 [SMILES: Cc1cc(N(CCCO)CC(=O)NCCc2ccc3c(c2)OCO3)nc(-n2ccnc2)n1]

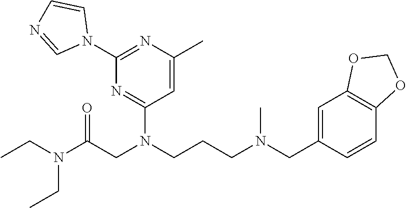 [SMILES: CCN(CC)C(=O)CN(CCCN(C)Cc1ccc2c(c1)OCO2)c1cc(C)nc(-n2ccnc2)n1]

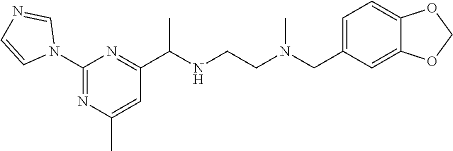 [SMILES: Cc1cc(C(C)NCCN(C)Cc2ccc3c(c2)OCO3)nc(-n2ccnc2)n1]

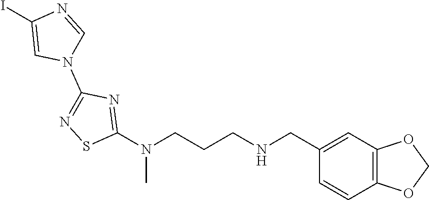 [SMILES: CN(CCCNCc1ccc2c(c1)OCO2)c1nc(-n2cnc(I)c2)ns1]

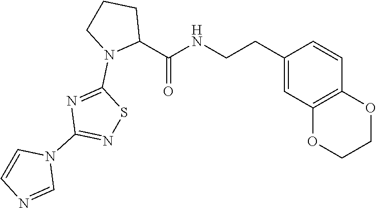 [SMILES: O=C(NCCc1ccc2c(c1)OCCO2)C1CCCN1c1nc(-n2ccnc2)ns1]

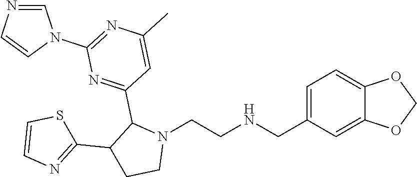 [SMILES: Cc1cc(C2C(c3nccs3)CCN2CCNCc2ccc3c(c2)OCO3)nc(-n2ccnc2)n1]

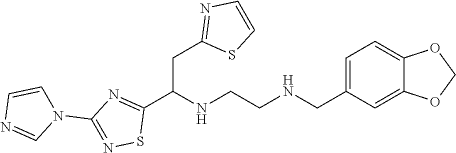 [SMILES: c1cn(-c2nsc(C(Cc3nccs3)NCCNCc3ccc4c(c3)OCO4)n2)cn1]

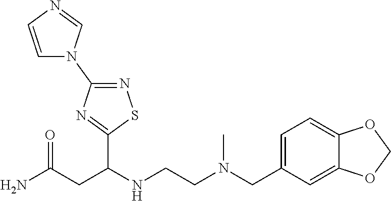 [SMILES: CN(CCNC(CC(N)=O)c1nc(-n2ccnc2)ns1)Cc1ccc2c(c1)OCO2]

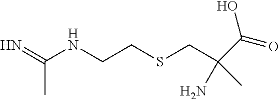

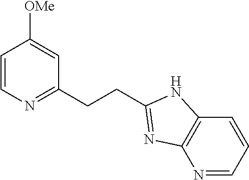 [SMILES: COc1ccnc(CCc2nc3ncccc3[nH]2)c1]

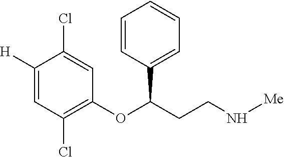 [SMILES: CNCC[C@@H](Oc1cc(Cl)ccc1Cl)c1ccccc1]

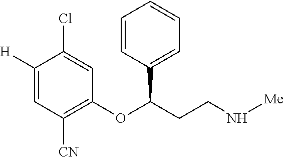 [SMILES: CNCC[C@@H](Oc1cc(Cl)ccc1C#N)c1ccccc1]

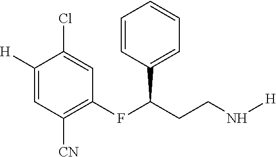

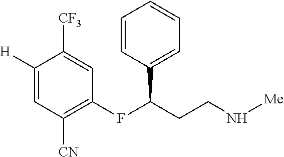

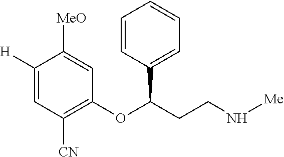 [SMILES: CNCC[C@@H](Oc1cc(OC)ccc1C#N)c1ccccc1]

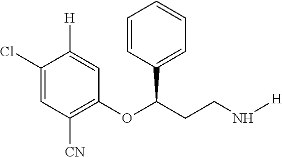 [SMILES: N#Cc1cc(Cl)ccc1O[C@H](CCN)c1ccccc1]

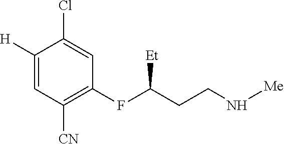

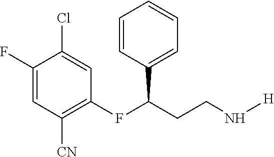

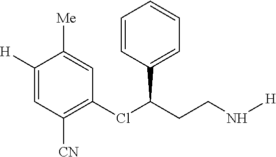

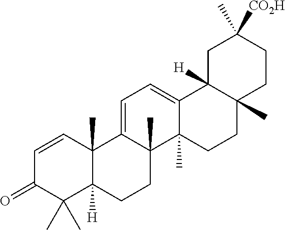

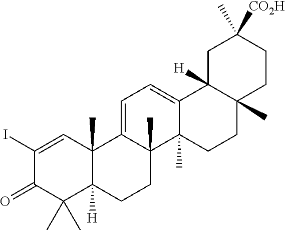

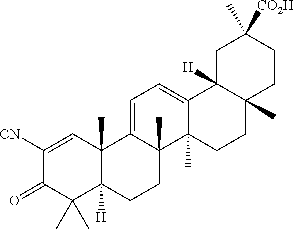

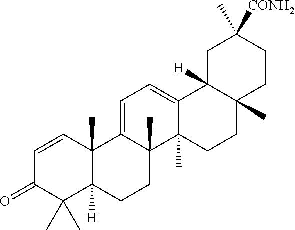

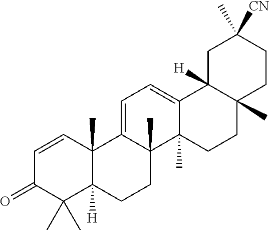

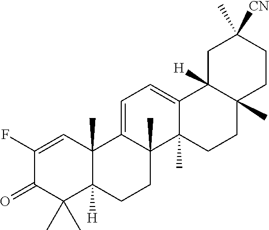

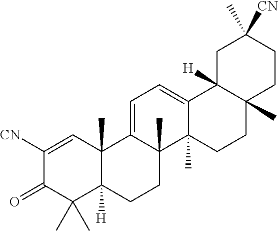

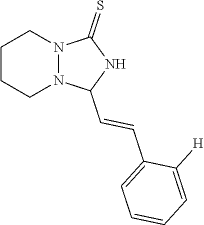 [SMILES: S=C1NC(/C=C/c2ccccc2)N2CCCCN12]

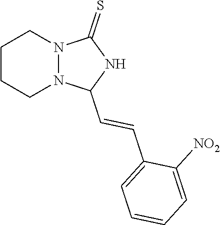 [SMILES: O=[N+]([O-])c1ccccc1/C=C/C1NC(=S)N2CCCCN12]

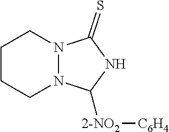

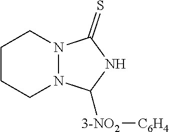

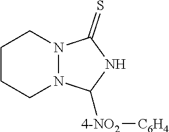

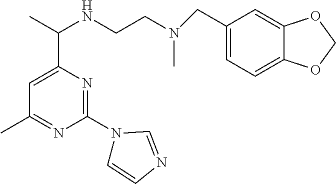 [SMILES: Cc1cc(C(C)NCCN(C)Cc2ccc3c(c2)OCO3)nc(-n2ccnc2)n1]

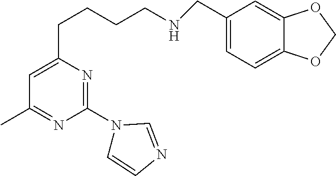 [SMILES: Cc1cc(CCCCNCc2ccc3c(c2)OCO3)nc(-n2ccnc2)n1]

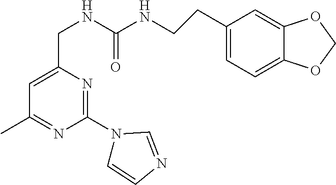 [SMILES: Cc1cc(CNC(=O)NCCc2ccc3c(c2)OCO3)nc(-n2ccnc2)n1]

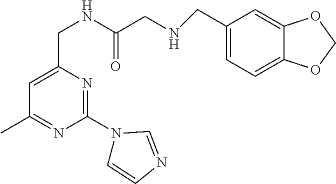 [SMILES: Cc1cc(CNC(=O)CNCc2ccc3c(c2)OCO3)nc(-n2ccnc2)n1]

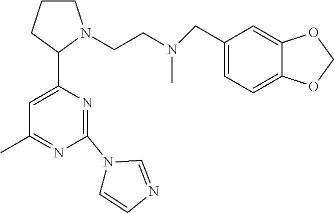 [SMILES: Cc1cc(C2CCCN2CCN(C)Cc2ccc3c(c2)OCO3)nc(-n2ccnc2)n1]

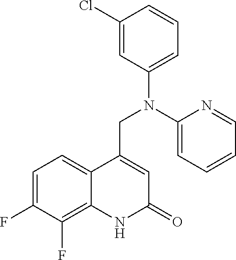 [SMILES: O=c1cc(CN(c2cccc(Cl)c2)c2ccccn2)c2ccc(F)c(F)c2[nH]1]

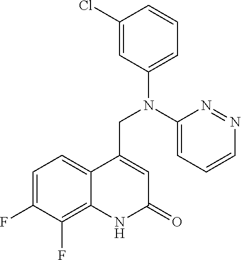 [SMILES: O=c1cc(CN(c2cccc(Cl)c2)c2cccnn2)c2ccc(F)c(F)c2[nH]1]

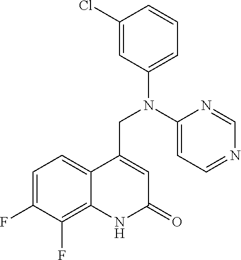 [SMILES: O=c1cc(CN(c2cccc(Cl)c2)c2ccncn2)c2ccc(F)c(F)c2[nH]1]

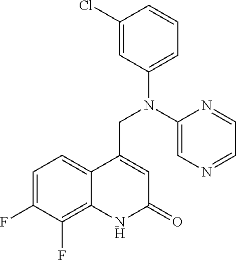 [SMILES: O=c1cc(CN(c2cccc(Cl)c2)c2cnccn2)c2ccc(F)c(F)c2[nH]1]

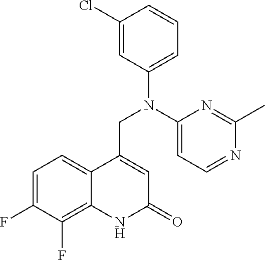 [SMILES: Cc1nccc(N(Cc2cc(=O)[nH]c3c(F)c(F)ccc23)c2cccc(Cl)c2)n1]

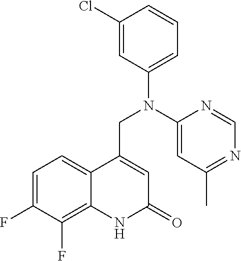 [SMILES: Cc1cc(N(Cc2cc(=O)[nH]c3c(F)c(F)ccc23)c2cccc(Cl)c2)ncn1]

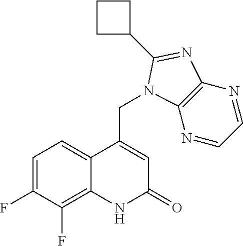 [SMILES: O=c1cc(Cn2c(C3CCC3)nc3nccnc32)c2ccc(F)c(F)c2[nH]1]

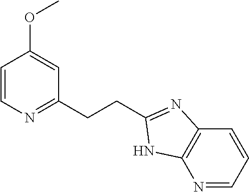 [SMILES: COc1ccnc(CCc2nc3cccnc3[nH]2)c1]

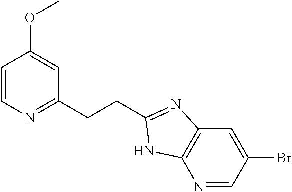 [SMILES: COc1ccnc(CCc2nc3cc(Br)cnc3[nH]2)c1]

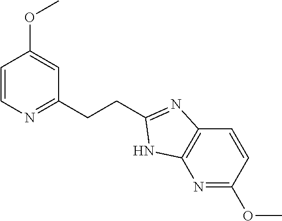 [SMILES: COc1ccnc(CCc2nc3ccc(OC)nc3[nH]2)c1]

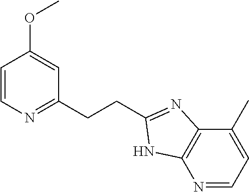 [SMILES: COc1ccnc(CCc2nc3c(C)ccnc3[nH]2)c1]

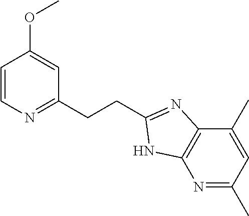 [SMILES: COc1ccnc(CCc2nc3c(C)cc(C)nc3[nH]2)c1]

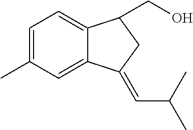 [SMILES: Cc1ccc2c(c1)/C(=C/C(C)C)CC2CO]

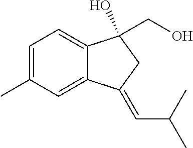 [SMILES: Cc1ccc2c(c1)/C(=C/C(C)C)C[C@]2(O)CO]

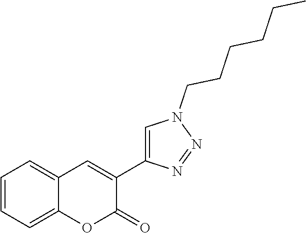 [SMILES: CCCCCCn1cc(-c2cc3ccccc3oc2=O)nn1]

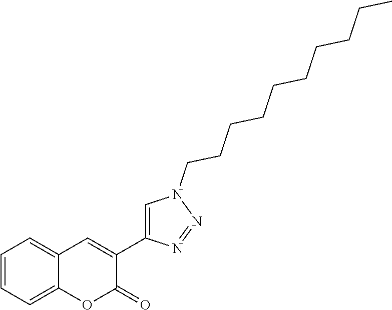 [SMILES: CCCCCCCCCCn1cc(-c2cc3ccccc3oc2=O)nn1]

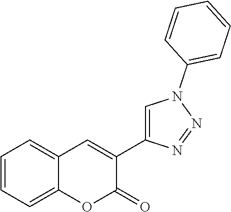 [SMILES: O=c1oc2ccccc2cc1-c1cn(-c2ccccc2)nn1]

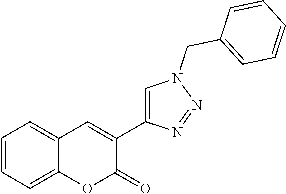 [SMILES: O=c1oc2ccccc2cc1-c1cn(Cc2ccccc2)nn1]

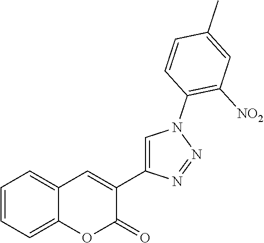 [SMILES: Cc1ccc(-n2cc(-c3cc4ccccc4oc3=O)nn2)c([N+](=O)[O-])c1]

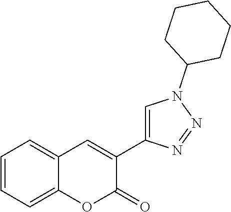 [SMILES: O=c1oc2ccccc2cc1-c1cn(C2CCCCC2)nn1]

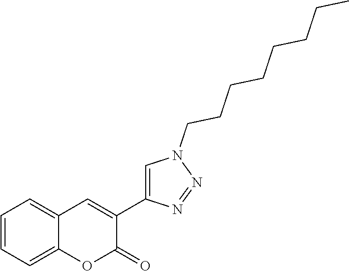 [SMILES: CCCCCCCCn1cc(-c2cc3ccccc3oc2=O)nn1]

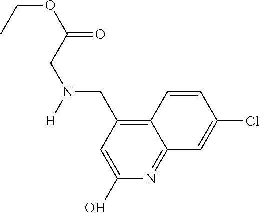 [SMILES: CCOC(=O)CNCc1cc(O)nc2cc(Cl)ccc12]

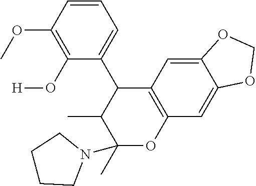 [SMILES: COc1cccc(C2c3cc4c(cc3OC(C)(N3CCCC3)C2C)OCO4)c1O]

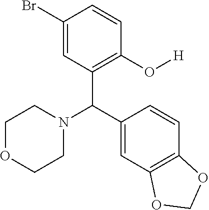 [SMILES: Oc1ccc(Br)cc1C(c1ccc2c(c1)OCO2)N1CCOCC1]

‘Pharmaceutically acceptable salts’ of compounds for use in the present invention include salts with inorganic bases, salts with organic bases, salts with inorganic acids, salts with organic acids and salts with basic or acidic amino acids. Exemplary salts include hydrochloride salt, acetate salt, trifluoroacetate salt, methanesulfonate salt, 2-hydroxypropane-1,2,3-tricarboxylate salt, (2R,3R)-2,3-dihydroxysuccinate salt, phosphate salt, sulphate salt, benzoate salt, 2-hydroxy-benzoate salt, S-(+)-mandelate salt, S-(−)-malate salt, S-(−) pyroglutamate salt, pyruvate salt, p-toluenesulfonate salt, 1-R-(−)-camphorsulfonate salt, fumarate salt and oxalate salt. The compound may be in either solvate (e.g. hydrate) or non-solvate (e.g. non-hydrate) form. When in a solvate form, additional solvents may be alcohols such as propan-2-ol.

The compounds described herein are useful in the prevention of viral replication and/or the prevention or treatment of a viral infection. The type of virus associated with this use is not particularly limited, but specifically includes rhinovirus, influenza virus (A and B), Avian flu, parainfluenza virus (1, 2 and 3), respiratory syncytial virus, adenovirus, coronavirus (e.g. SARS coronavirus), Epstein-Barr virus, enterovirus, metapneumovirus, adenovirus, measles virus, herpes simplex virus, varicella-zoster virus, ebola and cytomegalovirus. Preferably, the virus is rhinovirus, influenza virus, parainfluenza virus, respiratory syncytial virus, or SARS coronavirus. More preferably, the virus is influenza virus, parainfluenza virus, or respiratory syncytial virus (especially respiratory syncytial virus).

In view of the above viruses which may be targeted by the compounds described herein, a number of different medical conditions caused by viral infections may be prevented or treated. These conditions include influenza (i.e. flu), pharyngitis, rhinopharyngitis (i.e. common cold), laryngitis, gingivostomatitis, parotitis, pneumonia, bronchitis, bronchiolitis, laryngotracheobronchitis (i.e. croup), rhinitis, sinusitis, tonsillitis, tracheitis, measles, chicken pox, asthma exacerbation, chronic obstructive pulmonary disease (COPD) exacerbation, bronchiectasis exacerbation, cystic fibrosis exacerbation, otitis media, viral-associated wheeze, and severe acute respiratory syndrome (SARS). Preferably, the viral infection is influenza, rhinopharyngitis, pneumonia, asthma exacerbation, chronic obstructive pulmonary disease (COPD) exacerbation, or bronchiolitis, more preferably influenza, rhinopharyngitis, pneumonia, or bronchiolitis.

Previous studies have suggested that there is a link between NOS enzymes and the mediation of inflammatory conditions (see, for example, Stark et al. J. Infectious Diseases, 2005, 191, 387-395 or Esposito et al. Curr. Opin. in Invest. Drugs, 2007, 8, 899-909). However, it has been shown in clinical studies that selective iNOS inhibitors are not directly suitable for the prevention or treatment of inflammatory conditions such as asthma (Singh et al. Am. J. Respir. Crit. Care Med., 2007, 176, 988-993). The present invention therefore provides a new clinical strategy for the prevention or treatment of inflammatory conditions which are caused and/or exacerbated by viral infections.

The compound for use according to the invention may be presented in the form of a composition, comprising a compound as defined herein and one or more pharmaceutically acceptable excipients.

The one or more pharmaceutically acceptable excipients in the composition may include pharmaceutically acceptable diluents and carriers. Pharmaceutically acceptable diluents, excipients and carriers that may be used in the compositions include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, coenzyme A, cellulose-based substances, polyethylene glycol, sodium carboxymethylcellulose, polyacrylates, waxes, polyethylene-polyoxypropylene-block polymers, polyethylene glycol and wool fat.

The composition may also contain one or more additional active pharmaceutical ingredients. In particular, additional active pharmaceutical ingredients may include existing therapies used in the prevention of viral replication and/or the prevention or treatment of viral infections.

Examples of existing antiviral treatments suitable for use in combination therapy include abacavir, aciclovir, adefovir, amantadine, amprenavir, ampligen, arbidol, atazanavir, atripla, balavir, boceprevirertet, cidofovir, combivir, dolutegravir, darunavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, interferon, lamivudine, lopinavir, loviride, maraviroc, moroxydine, methisazone, nelfinavir, nevirapine, nexavir, novir, oseltamivir, peginterferon alfa-2a, penciclovir, peramivir, pleconaril, podophyllotoxin, raltegravir, ribavirin, rimantadine, ritonavir, pyramidine, saquinavir, sofosbuvir, stavudine, tea tree oil, telaprevir, tenofovir, tenofovir disoproxil, tipranavir, trifluridine, trizivir, tromantadine, truvada, valaciclovir (valtrex), valganciclovir, vicriviroc, vidarabine, viramidine, zalcitabine, zanamivir (relenza), and zidovudine.

As will be appreciated by those skilled in the art, further active pharmaceutical ingredients may be included in the composition in order to ameliorate the side-effects associated with any of the above existing antiviral treatments.

The compound or composition may be administered orally, parenterally, by inhalation spray, topically, rectally, nasally, buccally, vaginally or via an implanted reservoir. Preferably, the compositions are administered orally, nasally or by inhalation spray (preferably by aerosol delivery to the nose and lung). The composition may contain any conventional non-toxic pharmaceutically-acceptable carriers, adjuvants or vehicles. The term parenteral as used herein includes intraperitoneal, subcutaneous, intracutaneous, intravenous, intramuscular, intra-articular, intrasynovial, intrasternal, intrathecal, intraorbital, and intralesional injection or infusion techniques.

The composition may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

In the case of nasal aerosol delivery, the compound or composition may be formulated with an appropriate propellant and solvent. The formulation may then be pressurised in a canister. The formulation may be administered by individual sprays ejected from the canister via a metering valve upon activation by an actuator. The volume of the formulation ejected from the canister by a single spray can be adjusted by known methods, depending on the desired amount of active agent to be ejected per spray and the concentration of the active agent in the formulation. The formulation may also be delivered nasally or to the orpharynx using atomisers or nebulisers, including mesh nebulisers, ultrasonic and jet nebulisers. Delivery in a dry powder aerosol may also be used.

In the case of delivery to the oral cavity, pharynx, larynx and the airways and alveoli of the lung this may be accomplished using nebuliser, dry powder inhaler, metered dose inhaler, pressurised metered dose inhaler, with or without a spacer device, atomiser, administered either orally or nasally or if intubated via an endotracheal tube or tracheostomy tube. It may also be delivered via a spacer device or any other device that produces aerosolised particles of the drug.

In a further aspect of the invention, there is provided a method of preventing viral replication and/or preventing or treating viral infections in a subject comprising administering a prophylactically or therapeutically effective amount of a compound, or composition, according to the invention.

As will be appreciated by those skilled in the art, any of the preferred embodiments of the previous aspect of the invention are also applicable to this aspect of the invention.

The subject is preferably a mammal, such as a human or animal (preferably a human or bovine animal (e.g. cows), more preferably a human).

The invention will now be described in more detail by way of example only and with reference to the following figures.

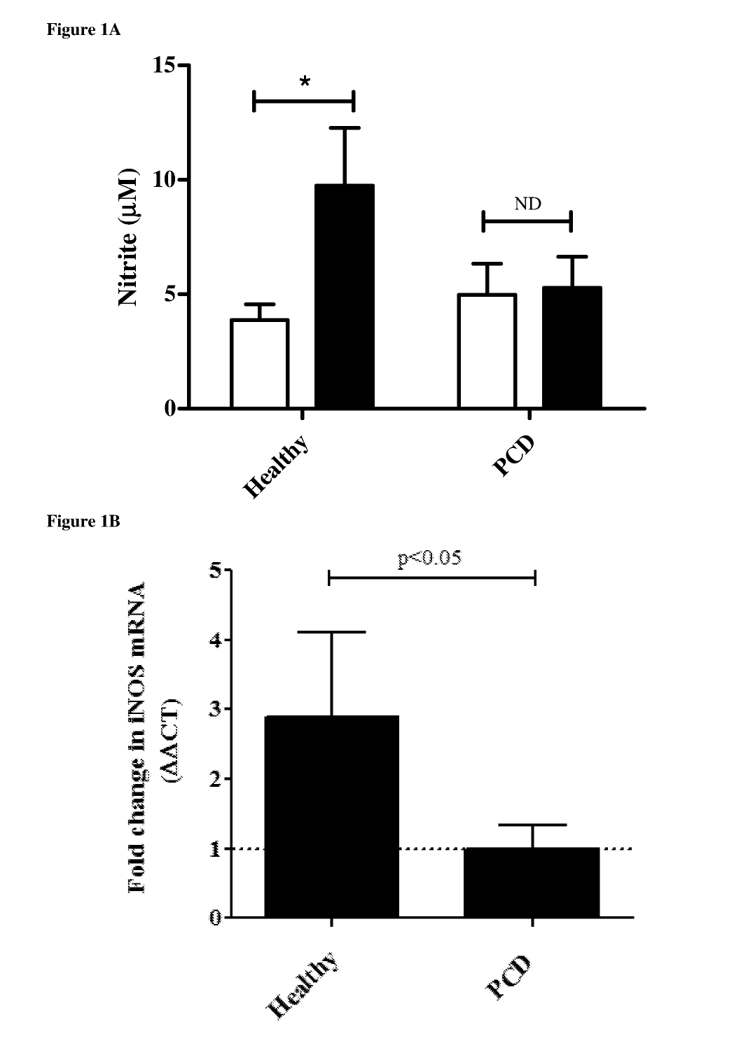

FIG. 1(A) Nitric oxide release (measured as nitrate and nitrite) of ciliated epithelial cells exposed to control (medium only) or RSV for 72 h. White bars represent control cells and black bars represent cells exposed to RSV. Dotted line represents the average medium only levels measured at time zero (n=12). (B) The fold change in inducible nitric oxide synthase (iNOS) gene NOS2 expression in human ciliated cells after incubation with RSV as determined by RT-qPCR. Data is expressed as the Log₂ relative gene expression using 2^{-ΔΔCT} and GAPDH as the housekeeping gene. Human ciliated cells from healthy subjects displayed a significant (P<0.05) increase in NOS2 gene expression (n=9), which was significantly different from epithelial cells obtained from PCD patients where no increase in NO or NOS2 expression was seen (n=4).

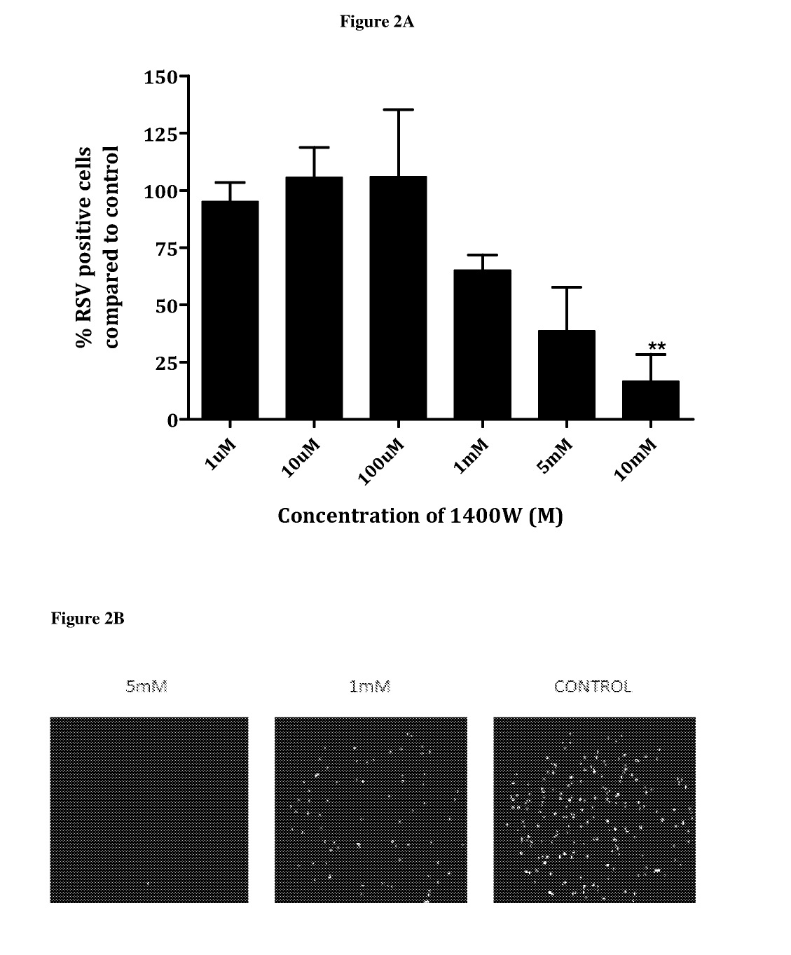

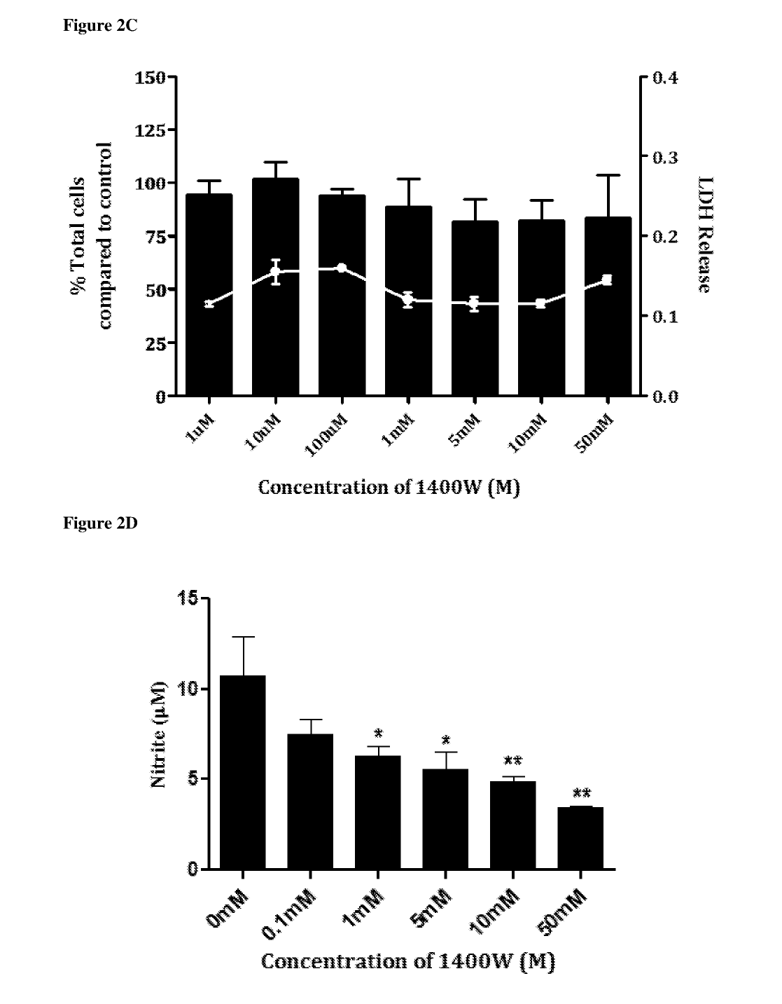

FIG. 2(A) The number of RSV infected A549 cells following incubation with different concentrations of the iNOS inhibitor 1400 W as a percentage of the control (no inhibitor). Significant changes are highlighted by * (p<0.05), **(p<0.01). (B) Immunofluorescence images of RSV infected A549 cells with 5 mM, 1 mM and 0 mM 1400 W. (C) The concentration of 1400 W was not toxic to the RSV infected cells as measured by the amount of the cytoplasmic enzyme lactate dehydrogenase (LDH) in the cell culture supernatant and the number of cells remaining attached to the well. The experiment was performed six times (n=6). (D) Nitric oxide production by respiratory epithelial cells exposed to the iNOS inhibitor 1400 W and in response to infection by RSV.

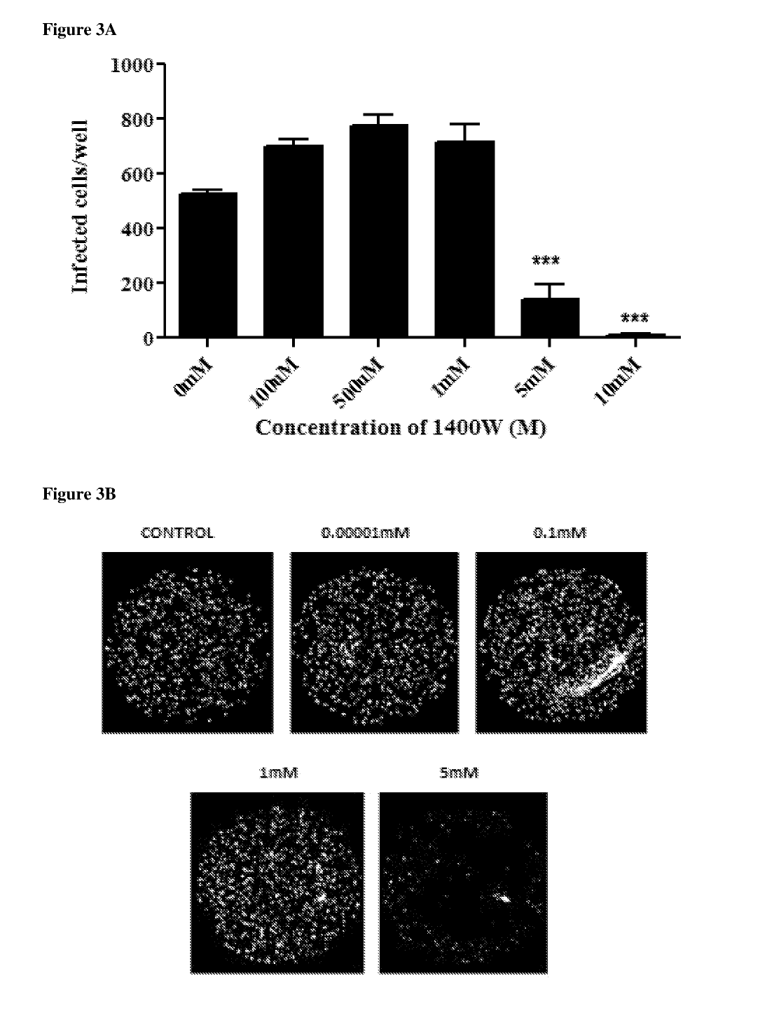

FIG. 3(A) The number of influenza infected A549 cells following incubation with different concentrations of the iNOS inhibitor 1400 W as a percentage of the control (no inhibitor). Significant changes are highlighted by * (p<0.05), **(p<0.01). (B) Immunofluorescence images of influenza infected A549 cells exposed to 1400 W or control.

### EXAMPLES

### Materials and Methods

### Respiratory Epithelial Cell Culture

Human ciliated epithelium was obtained by brushing the inferior nasal turbinate with a 2-mm cytology brush (Keymed, Southend-on-Sea, UK) as previously described [1]. All individuals gave their consent to be included in the study and all samples were obtained with the individual's permission and with ethical approval by the Leicestershire Ethical Review Committee. The sample was vigorously pipetted into 2 ml 20 mM Hepes-buffered medium 199 (pH 7.4) (Gibco Life Technologies, UK), containing penicillin (100 IU/ml), streptomycin (100 μg/ml) and fungizone (2.5 μg/ml) to breakup large cell clumps and kept at 4° C. overnight. 1 ml was then placed in a collagen coated well of a 12-well plate (Nunclon, UK) together with 1 ml of basal epithelial growth media (BEGM), containing penicillin (100 IU/ml), streptomycin (100 μg/ml) and fungizone (2.5 μg/ml), at 37° C. The basal cells were fed every 2-3 days by using 1 ml BEGM containing antibiotics. When the cells were >90% confluent the cells were detached using Trypsin/EDTA (Sigma, UK) for 5 min. The cells were then centrifuged (4,000×g for 10 min) and the supernatant was removed. The pellet was resuspended in BEGM to a concentration of 1×10⁶ cells/ml. 400 al of cell suspension was added to each well of an 24-well plate (Corning, Costar) and grown until at least 80% confluent.

The remaining basal cells were seeded on collagen-coated, semipermeable membrane supports (Transwell-Col; 12 mm in diameter; 0.4 am pore size; Corning-Costar, Coming, N.Y.) as previously described [2]. At confluence, the apical medium was removed and the cells were maintained at an air-liquid interface (ALI) to allow differentiation of the epithelial subtypes. Well differentiated cultures, were studied approximately 4 to 6 weeks after initiation of an ALI unless otherwise stated.

A549 cells (American Type Culture Collection (ATCC), Manassas, Va.) were grown in RPMI medium (Gibco) with 10% heat-inactivated foetal calf serum (Sigma), pen/strep and fungizone.

### Virus Strains and Growth Conditions

Wild-type RSV (A2) Long strain stocks were prepared in monolayers of BSC-1 monkey kidney cells (MOI 0.01). Infected cells were incubated for 7-10 days in antibiotic free GMEM-NEAA supplemented with 2% Foetal Calf Serum at 5% CO₂, 37° C. Stocks were harvested by disruption with glass beads for 1 minute and the supernatant was centrifuged at 1000 g for 5 minutes to remove cell debris. The filtrate was then purified by centrifugation through a polyethersulphone membrane containing a pore size of 1000 000 Daltons MWCO (1000 kD) (Vivaspin-20, Vivascience, Gloucester, UK) as previously described [3]. The virus fractions were collected and pooled in BEBM (Lonza), and aliquots were stored at −80° C. containing about 1×10⁵ PFU/ml.

Human influenza virus A/Puerto Rico/8/34 (H1N1) (PR8) was grown in 10-day old fertilized chicken eggs. After incubation at 37° C. for 2 days, the allantoic fluid was harvested and used for infection. Allantoic fluid was harvested from uninfected chicken eggs for use as a negative control. Viral stocks were titred by plaque assay using Madin-Darby canine kidney (MDCK) cells grown to 90% confluency in 96-well dishes. Cells were washed with PBS and infected with serial dilutions of the virus in Dulbecco's modified eagle medium (DMEM) with 10% foetal calf serum, penicillin 100 U/ml, and streptomycin 10 μg/ml for 1 h at 37° C. The inoculum was removed and cells were incubated with 200 μl DMEM (medium containing 1.4% BSA, 2 μg/ml of trypsin and antibiotics) at 37° C., 5% CO₂ for 2-3 days. Virus plaques were visualized by staining with mouse anti-HA antibodies and a fluorescent Alexa-594 labelled secondary anti-mouse antibody (Invitrogen, UK).

### Viral Infection of Primary Epithelial Cell Cultures

Frozen aliquots of RSV were thawed immediately prior to use. Three day old tissue culture medium was removed and stored at −70° C., 400 μl of viral suspension (MOI of 1) was then applied for 1 h at 37° C. Control wells received BEBM alone. After this time the virus was removed and cells were fed with fresh BEGM. The infection was then allowed to continue for a further 72 h. After this time the supernatants were harvested and stored at −70° C. for cytokine analyses. Cells were fixed overnight with 4% paraformaldehyde in phosphate buffered saline (PBS) for immunostaining.

### Ciliary Beat Frequency and Beat Pattern

To determine ciliary beat frequency, both the brain slices and the respiratory cells in culture were placed in an incubation (37° C.) chamber and were observed via an inverted microscope system (Nikon TU1000, UK). Tissue was allowed to equilibrate for 30 min before readings. Beating cilia were recorded using a Troubleshooter or Motion Pro X4 digital high-speed video camera (Lake Image Systems, USA) at a rate of 250 frames per second using an ×40 objective as previously described [1]. For each experimental condition, five readings of ciliary beat frequency (CBF) were taken from different areas along each ciliated edge. This was converted to CBF by a simple calculation (CBF=250/(number frames for 5 beats)×5) [1]. Ciliary amplitude was also measured as described previously [4].

### Cytotoxicity Assay

To determine the level of cell damage following infection, 50 μl of the culture supernatant from each well were transferred to the corresponding well in 96 well microtitre plate. The LDH activity of the supernatant was measured by a LDH Assay kit, according to the manufacturer's instructions (Sigma, UK). 100 μl of LDH Assay solution was added to each well and the plate was incubated for 30 min at room temperature. After incubation, the absorbance was measured at two wavelengths, 490 nm (measurement) and 690 nm (reference), using a microplate reader (BioRad Laboratories, Hercules, Calif., USA). The percent LDH release was calculated by measuring the LDH content of lysed cells that remained attached to the plate.

### Chemokine and Cytokine and Nitric Oxide Analysis

Chemokines and cytokines were measured using a 96-well multispot assay (Meso Scale Discovery [MSD], Maryland, USA) according to the manufacturer's instructions. Cytokines were measured using a human Th1/Th2 standard 10 spot plate and human chemokines were measured using a high band MS6000 10 spot plate, using SECTOR Imager 6000 (MSD, Maryland, USA). The lower limit of detection was 1 pg/ml.

Nitric oxide was measured using a chemiluminescence analyser (model 280; Sievers Instruments; Boulder, USA) as previously described [5]. Briefly, 5-10 ul of culture supernatant was injected into the analyzer, where NO₃⁻ and NO₂⁻ are reduced by vanadium (III) chloride (in IM HCl) to nitric oxide. Once this mixes with ozone it emits a photon that is detected by a photomultiplier. A 100 mM nitrate solution was used to prepare a standard curve.

### Immunofluoresence Microscopy

Fixed cells were blocked by incubation in PBS containing BSA (3%) for 10 minutes and then stained for RSV antigens using 1:40 dilution of goat anti-RSV antibody (Abcam) in 1% BSA for 2 h. Unbound antibody was washed using 3 changes of PBS and bound antibody was detected using the secondary mouse anti-goat conjugated to FITC (Sigma, UK) diluted to 1:250 in 1% BSA. Nuclei were stained using Hoechst 33258 (Invitrogen). Cells were mounted with 50% glycerol in PBS, containing 0.01% n-propyl gallate. Low magnification images were obtained with a Nikon TU1000 fluorescence inverted microscope equipped with a Hamamatsu digital camera. High resolution optical sections were obtained using a Leica DMI 6000 CS fluorescence confocal inverted microscope and ×63 immersion oil lens. Images acquired by confocal microscopy were rendered by Imaris Software (Bitplane AG) using the blend or MIP filters.

### RNA Extraction, Retrotranscription and Quantitative Real Time RT-PCR

RNA was extracted by using High Pure Total RNA Isolation System Kit (Roche) according to the manufacturer's instructions. Samples were immediately processed for retrotranscription which was carried out by using the Transcriptor First Strand cDNA Synthesis Kit (Roche) according to the manufacturer's instructions. Briefly, annealing was performed at 25° C. for 10 min, extension at 37° C. for 1 h and inactivated at 70° C. for 15 min. Quantitative real time PCR was performed as previously described [6] in a Light Cycler apparatus (Roche) by using the Light Cycler DNA-Master SYBR Green I Kit (Roche). As PCR template, 5 μl of cDNA was used. Primer efficiency was verified by using serial dilution of cDNA ranging from 10² to 10⁶ target copies per reaction (10⁴-10⁸ target copies per sample), and only oligonucleotides with comparable efficiency were chosen. Primers spanning 100-150 bp segments for B actin, GAPDH, iNOS, nNOS, eNOS.

### Effect of NOS Inhibitors and NO Donors on RSV and Influenza Virus Replication in Human Respiratory Epithelial Cells

A549 cells were grown to confluence in 96-well microtitre plates (Corning). The epithelial cells were then infected with 1×10⁵ pfu RSV or influenza virus for 1 hour and then removed. Cells were then overlaid with RPMI containing L-NAME (Sigma), 1400 W (Sigma), SNAP, SIN-1, L-arginine (Sigma), BYK191023 (Tocris Bioscience), or media alone and incubated at 5% CO₂, 37° C. for 24 h. Cells were fixed with an equal mix of methanol:acetone for 15 minutes at room temperature and stained for viral antigen (as above).

### Mouse Infection Model

Groups of 5 nine week old female balb/c mice (HarlanOlac, Bicester, UK) were infected intranasally with 1×10⁵ pfu/50 ul RSV. This dose does not to cause disease in mice but viral replication can be detected in the lungs. Mice were then treated with either PBS or 10 mg/kg 1400 W intraperitoneally every 12 h. Three and five days post infection five mice from each group were killed by cervical dislocation and the lungs were harvested into 10 ml of sterile PBS, weighed, and homogenized. Viable counts in lung homogenates and blood were determined by serial dilution in sterile PBS and plaque assay. All animal work was conducted in accordance with the UK national regulations.

### Results

Respiratory Epithelial Cells from Patients with PCD Displayed Reduced Staining for RSV Antigens after 72 h

To investigate the spread of viral antigen in ciliated epithelial cells from PCD patients and healthy volunteers the cells were fixed 72 hours post-infection and stained with antibodies specific for cilia (acetylated-tubulin) and RSV protein G, which are present on the surface of infected cells. Non-ciliated cells, including Clara cells and goblet cells, remained unstained. It was found that healthy cells displayed positive staining for RSV antigen (green) was observed on the apical surface of the cell and on the ciliary axoneme, which was confirmed by the co-localisation of RSV antigen (green) and acetylated tubulin antigen (red), producing a yellow colour, indicating preferential infection of ciliated cells (observation, not quantified). No viral antigens were observed in the control (uninfected) wells (data not shown). However, cells from patients with PCD displayed a reduced staining with RSV despite the presence of many ciliated cells. To determine whether RSV antigen staining corresponded with the level of viral progeny, the titre of virus released into the apical fluid of infected cell cultures after 72 h was measured. This showed that cells from PCD patients release significantly fewer infectious RSV particles into the apical fluid of infected cultures compared to healthy cell cultures.

Respiratory Cells from PCD Patients Show No Increase in Nitric Oxide or NOS2 Expression Following Infection with RSV

The production of nitric oxide by differentiated respiratory epithelial cells (ALI cultures) was measured in the apical supernatant by washing the cells 72 hrs after infection with RSV. It was found that the concentration of NO produced by healthy epithelial cells increased after incubation with RSV, which was not seen following infection of PCD patient's ciliated culture (FIG. 1A). To verify the finding of decreased NO biosynthesis in ciliated cultures from PCD patients, the inducible nitric oxide synthase (iNOS) gene expression in both healthy and PCD ciliated epithelial cells using RT-qPCR was analysed. It was found that in healthy individuals the expression of the iNOS gene NOS2 increased threefold following infection with RSV. However, cells from patients with PCD did not increase from the baseline (FIG. 1B).

The Specific iNOS Inhibitor 1400 W Reduces RSV Replication in A549 Respiratory Epithelial Cells

To investigate whether an iNOS inhibitor could reduce RSV replication in healthy human respiratory epithelial cells, RSV infected A549 epithelial cells were exposed to the specific iNOS inhibitor 1400 W for 24 hrs. After this time the number of infected cells was counted using immunofluorescence staining and the level of cell toxicity was determined.

It was found that the number of RSV-infected cells significantly decreased at concentrations above 1 mM 1400 W (FIGS. 2A and 2B). To determine whether the concentration series of 1400 W we used was toxic to the A549 cells, and to eliminate the possibility that the reduction in viral antigen was due to toxicity, the concentration of lactate dehydrogenase (LDH) in the cell culture supernatant was measured. LDH is an enzyme that is only released by cells when the cell membrane integrity is reduced. Therefore the concentration of LDH in the cell culture supernatant is directly correlated to cell death. It was found that the amount of LDH in the cell culture supernatant of uninfected and RSV infected cells was not affected by the concentration of 1400 W they were exposed to (FIG. 2C)

The Specific iNOS Inhibitor 1400 W Reduced the Increase in NO Production in A549 Cells Following RSV Infection

It was found that RSV infected A549 epithelial cells exposed to concentrations of 1400 W above 1 mM showed a significant decrease in the amount of NO released into the cell culture supernatant (FIG. 2D). Supernatant analysed from RSV-infected cells overlaid with water in dilution similar to the preparation of 1400 W showed no change in the levels of NO in the cell culture supernatant.

### The Effect of NOS Inhibitors Including 1400 W on RSV and Influenza Virus Replication can be Seen in Table II.

RSV replication in lungs of infected mice is significantly reduced with 1400 W To gauge the importance of iNOS function during RSV replication in vivo, mice with and without 1400 W administration prior to RSV infection were examined. Mice were injected i.p. every 12 h with 1400 W or saline for 24 h, followed by intranasal inoculation with RSV as described in Materials and Methods. Three and five days later lungs were harvested and the viral loads determined. The titre of infectious RSV in the lungs of 1400 W-treated mice was significantly lower when compared with saline-treated mice after 3 days (see Table III). Treatment with 1400 W and RSV also resulted in significantly reduced total lung nitrite compared with saline-treated and RSV infected mice. This indicates that treatment with 1400 W reduced total lung NOS activity in these mice.

### Discussion

It has been recently discovered that cultured respiratory epithelial cells from patients with PCD are more resistant to infection with respiratory syncytial virus (RSV) compared to epithelial cells from healthy individuals. RSV is the most important respiratory pathogen causing lower respiratory tract infections worldwide. The WHO estimates that RSV causes 64 million infections and 160,000 deaths annually. This finding therefore provides a new antiviral or new therapeutic strategy against viruses such as RSV.

Patients with PCD exhibit extremely low levels of exhaled and nasal nitric oxide (NO). Whether the low viral replication was related to low NO exhibited in these patients was investigated. In healthy epithelial cells, NO is produced from L-arginine by three mammalian isoforms of nitric oxide synthase (NOS): neuronal NOS (nNOS), inducible NOS (iNOS) and endothelial NOS (eNOS) all of which are expressed within the respiratory tract. Previous studies have suggested a link between low NO and reduced iNOS activity and levels of iNOS and eNOS have both been shown to be very low in children with PCD. This would be consistent with the findings on the clinical effects of selective iNOS inhibitors on exhaled nitric oxide, which show that the majority derives from the iNOS isoform.

To determine whether there is a link between low levels of NO and RSV replication, epithelial cells from healthy individuals were incubated with the iNOS specific inhibitors 1400 W and BYK191023. Surprisingly, it was found found that compounds strongly inhibited RSV replication, which correlated with a reduction in NO production by these cells. In contrast, the non-iNOS specific inhibitor L-NAME did not inhibit viral replication. Furthermore the NO-generating compound S-nitroso-N-acetylpenicillamine (SNAP) and 3-morpholinosydnonimine hydrochloride (SIN-1), a peroxynitrite donor, did not affect viral replication.

Spermine NONOate, which rapidly generates NO in aqueous solution was unexpectedly shown to inhibit viral replication indicating that the effect of selective iNOS inhibition is not simply a consequence of decreased NO levels.

TABLE I
The chemokine and cytokine response from human epithelial cells infected with RSV for 72 hours

| Chemokine | | | | | | | | | |
|---|---|---|---|---|---|---|---|---|---|
| Cytokine | | Healthy (n = 5) | | | | PCD (n = 4) | | | |
| (pg/ml) | | Control | | RSV | | Control | | RSV | |
| Th1 | IFN-γ | 24 | (17-79) | 61 | (30-107) | 69 | (57-109) | 90 | (46-160) |
| | IL-1β | 4 | (4-96) | 19 | (15-36) | 17 | (12-27) | 23 | (14-36) |
| | IL-2 | 4 | (3-15) | 10 | (8-13) | 34 | (19-53) | 17 | (11-28) |
| | IL-12p70 | 4 | (3-26) | 10 | (8-23) | 19 | (9-46) | 15 | (9-28) |
| | TNFα | 17 | (12-106) | 71 | (24-156) | 120 | (82-165) | 112 | (71-138) |
| Th2 | IL-4 | 3 | (1-7) | 6 | (3-10) | 5 | (3-7) | 8 | (4-15) |
| | IL-5 | 5 | (3-59) | 22 | (18-52) | 17 | (13-31) | 19 | (14-30) |
| | IL-6 | 29 | (17-263) | 422 | (307-1989) | 1983 | (695-5000) | 1607 | (859-4868) |
| | IL-10 | 5 | (4-38) | 31 | (11-117) | 53 | (36-87) | 33 | (30-103) |
| | IL-13 | 13 | (7-48) | 47 | (10-84) | 68 | (46-90) | 58 | (30-69) |
| CCL11 | Eotaxin | 145 | (103-326) | 224 | (188-416) | 769 | (544-1558) | 321 | (260-373) |
| CCL4 | MIP-1β | 2 | (1-35) | 19 | (16-51) | 45 | (28-178) | 46 | (32-69) |
| CCL26 | Eotaxin-3 | 210 | (159-8302) | 2857 | (758-14826) | 16897 | (6878-25046) | 11979 | (4547-21213) |
| CCL17 | TARC | 71 | (44-277) | 173 | (123-327) | 595 | (354-1290) | 289 | (211-358) |
| CCL2 | MCP-1 | 12 | (9-40) | 156 | (26-308) | 211 | (190-452) | 367 | (71-391) |
| CCL22 | MDC | 183 | (181-633) | 527 | (348-1146) | 1770 | (1058-3230) | 558 | (485-663) |
| CCL13 | MCP-4 | 86 | (52-667) | 418 | (221-1013) | 739 | (480-1378) | 419 | (267-539) |
| CXCL8 | IL-8 | 31 | (13-3720) | 4557 | (519-7131)** | 2688 | (444-5184) | 3845 | (2014-5661) |
| CXCL10 | IP-10 | 107 | (92-508) | 426 | (218-3283) | 1372 | (924-2672) | 1468 | (878-2318) |

Significant changes from matched control are highlighted in boldface; two way ANOVA.
§ denotes significant difference between healthy and PCD.
P < 0.05.

TABLE II
The IC50 of different NOS inhibitors and NO donors on RSV and
influenza virus replication by A549 epithelial cell cultures

| | RSV | Influenza | |
|---|---|---|---|
| | RPMI | RPMI | DMEM |
| IC50¹ | (1.15 mM L-Arg) | (1.15 mM L-Arg) | (0.35 mM L-Arg) |
| NOS inhibitors | | | |
| L-NAME | No effect | No effect | nd |
| BYK191023 | 1.1 ± 0.5 mM | 4.3 ± 0.8 mM | 4.8 ± 0.4 mM |
| 1400W | 1.1 ± 0.5 mM | 4.6 ± 0.9 mM | 3.7 ± 1.0 mM |
| NO donors | | | |
| Spermine | 1.5 ± 0.5 mM | 0.6 ± 0.4 mM | nd |
| (NONOate) | | | |
| L-arginine | No inhibitory | | |
| | effect | | |

¹Concentration needed to give a 50% reduction in number of infected cells/well ± 95% CI after 24 h.
nd = not done

TABLE III
The viral load and nitrite concentration of the lungs of mice infected with RSV

| | Day of | Log₁₀ pfu | Nitrite (uM) lung |
|---|---|---|---|
| Treatment | Analysis | per gram lung | homogenate^{a} |
| Mock infected + PBS treated | 3 | <0.18 | 25.1 (4.1) |
| Mock infected + 1400W treated | 3 | <0.18 | 26.0 (5.4) |
| RSV infected + PBS treated | 3 | 1.26 ± 0.62 | 30.0 (2.0) |
| RSV infected + 1400W treated | 3 | 0.51 ± 0.17** | 16.7 (1.9)* |
| Mock infected + PBS treated | 5 | <0.18 | 35.1 (5.1) |
| Mock infected + 1400W treated | 5 | <0.18 | 21.6 (3.0) |
| RSV infected + PBS treated | 5 | 1.29 ± 0.91 | 25.5 (4.7) |
| RSV infected + 1400W treated | 5 | 1.18 ± 0.92 | 23.7 (3.3) |

Significant changes from RSV infected + PBS treated are highlighted in boldface and *; two way ANOVA.

### REFERENCES

[1] Chilvers, M. A. and C. O'Callaghan, Analysis of ciliary beat pattern and beat frequency using digital high speed imaging: comparison with the photomultiplier and photodiode methods. Thorax, 2000. 55(4): p. 314-7.
[2] Hirst, R. A., et al., Ciliated Air-Liquid Cultures as an Aid to Diagnostic Testing of Primary Ciliary Dyskinesia. Chest, 2010. 138(6): p. 1441-1447.
[3] Bataki, E. L., G. S. Evans, and M. L. Everard, Respiratory syncytial virus and neutrophil activation. Clin Exp Immunol, 2005. 140(3): p. 470-7.
[4] O'Callaghan, C. L., et al., The effect of viscous loading on brain ependymal cilia. Neuroscience Letters, 2008. 439(1): p. 56-60.
[5] Tsang, K. W., et al., Exhaled and sputum nitric oxide in bronchiectasis: correlation with clinical parameters. Chest, 2002. 121(1): p. 88-94.
[6] Oggioni, M. R., et al., Antibacterial activity of a competence-stimulating peptide in experimental sepsis caused by Streptococcus pneumoniae. Antimicrob Agents Chemother, 2004. 48(12): p. 4725-32.

1. A compound for use in the prevention of viral replication and/or the prevention or treatment of a viral infection, wherein the compound is a selective inhibitor of inducible nitric oxide synthase.

2. The compound for use according to claim 1, wherein the compound is of the formula: 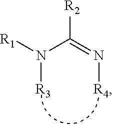
wherein
R₁ is hydrogen, an optionally substituted C₁₋₆ alkyl group, an optionally substituted C₂₋₆ alkenyl group, an optionally substituted C₆₋₁₀ aryl group, an optionally substituted C₇₋₁₆ aralkyl group, an optionally substituted 5- to 10-membered heterocyclyl group, or a group of the formula: 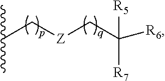
wherein
Z is an optionally substituted C₆₋₁₀ arylene group, an optionally substituted C₁₋₆ alkylene group, an optionally substituted C₂₋₆ alkenylene group, an optionally substituted 5- to 10-membered heterocyclylene group, a group of the formula —S(O)ₓ—, where x is 0, 1, or 2, a group of the formula —NR₈—, where R₈ is hydrogen, a C₁₋₆ alkyl group, or a C₆₋₁₀ aryl group, or a group of the formula —O—;
p is an integer from 0 to 5;
q is an integer from 0 to 5;
R₅ is hydrogen, an optionally substituted C₁₋₆ alkyl group, or an optionally substituted C₁₋₆ alkoxy group;
R₆ is a carboxyl group, an optionally substituted C₁₋₆ alkyl carbonyloxy group, an optionally substituted C₁₋₆ alkyl carbonyl group, an optionally substituted C₁₋₆ alkoxy carbonyl group, a carbamoyl group, or an optionally substituted C₁₋₆ alkyl carbamoyl group; and
R₇ is an optionally mono- or di-substituted amino group or an optionally substituted C₁₋₆ alkoxy group;
R₂ is hydrogen, an optionally substituted C₁₋₆ alkyl group, an optionally substituted C₂₋₆ alkenyl group, an optionally substituted C₂₋₆ alkynyl group, an optionally substituted C₃₋₆ cycloalkyl group, an optionally substituted C₃₋₆ cycloalkyl-C₁₋₆ alkyl group, an optionally substituted C₇₋₁₆ aralkyl group, or an optionally substituted C₆₋₁₀ aryl group;
R₃ is hydrogen, an optionally substituted C₁₋₆ alkyl group, or an optionally substituted C₆₋₁₀ aryl group; and
R₄ is hydrogen, an optionally substituted C₁₋₆ alkyl group, or an optionally substituted C₆₋₁₀ aryl group;
or R₃ and R₄ are joined together to form an optionally substituted 5- to 10-membered monocyclic or bicyclic heterocyclyl group;
provided that R₁, R₂, R₃, and R₄ are not all hydrogen;
or a pharmaceutically acceptable salt thereof.

3. The compound for use according to claim 2, wherein
R₁ is a group of the formula: 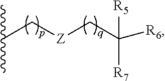
wherein
Z is (i) a C₆₋₁₀ arylene group optionally substituted by 1 to 3 substituents independently selected from halogen, a C₁₋₆ alkyl group, and a C₁₋₆ alkoxy group, (ii) a C₂₋₆ alkenylene group optionally substituted by 1 to 3 substituents independently selected from halogen and a C₁₋₆ alkyl group, (iii) a group of the formula —S(O)ₓ—, where x is 0, 1, or 2, (iv) a group of the formula —O—, or (v) a C₁₋₃ alkylene group;
p is 0, 1, or 2;
q is 0, 1, or 2;
R₅ is hydrogen or a C₁₋₆ alkyl group;
R₆ is a carboxyl group, a C₁₋₆ alkoxy carbonyl group, a carbamoyl group optionally substituted by a 5- or 6-membered heterocyclyl group, or a C₁₋₆ alkyl carbamoyl group; and
R₇ is an amino group optionally mono- or di-substituted by a C₁₋₆ alkyl group or a C₇₋₁₀ aralkyl group.

4. The compound for use according to claim 2 or claim 3, wherein
R₂ is hydrogen, or a C₁₋₆ alkyl group, a C₂₋₆ alkenyl group, a C₂₋₆ alkynyl group, or a C₃₋₆ cycloalkyl-C₁₋₆ alkyl group, each of which is optionally substituted by 1 to 3 substituents independently selected from:
(i) —CN;
(ii) —NO₂;
(iii) a group of the formula —COR⁸, wherein R⁸ is hydrogen, a C₁₋₆ alkyl group, a group of the formula —OR⁹, wherein R⁹ is hydrogen or C₁₋₆ alkyl, or a group of the formula NR¹⁰R¹¹, wherein R¹⁰ and R¹¹ are independently selected from hydrogen or a C₁₋₆ alkyl group;
(iv) a group of the formula —S(O)ₘR¹², wherein m is 0, 1 or 2, R¹² is hydrogen, a C₁₋₆ alkyl group, hydroxy or a group of the formula NR¹³R¹⁴ wherein R¹³ and R¹⁴ are independently hydrogen or a C₁₋₆ alkyl group; (v) a group of the formula PO(OR¹⁵)₂, wherein R¹⁵ is hydrogen or a C₁₋₆ alkyl group;
(vi) a group of the formula NR¹⁶R¹⁷, wherein R¹⁶ and R¹⁷ are independently selected from hydrogen, a C₁₋₆ alkyl group, a group of the formula —COR¹⁸, wherein R¹⁸ is hydrogen or a C₁₋₆ alkyl group, or a group of the formula —S(O)_{m′}R¹⁹, wherein m′ is 0, 1 or 2 and R¹⁹ is hydrogen or a C₁₋₆ alkyl group;
(vii) a halogen atom; and
(viii) a group of the formula —OR²⁰, wherein R²⁰ is hydrogen, a C₁₋₆ alkyl group optionally substituted by 1 to 3 halogen atoms, a C₆₋₁₀ aryl group or a group of the formula —COR²¹, wherein R²¹ is hydrogen or a C₁₋₆ alkyl group.

5. The compound for use according to claim 2 or claim 3, wherein
R₂ is hydrogen, or a C₁₋₆ alkyl group optionally substituted by a C₆₋₁₀ aryl group or a 5- to 7-membered heterocyclyl group, each of which is optionally substituted by 1 to 3 substituents independently selected from:
(i) —NH₂;
(ii) —OH;
(iii) a halogen atom;
(iv) a C₁₋₆ alkyl group; and
(v) a C₁₋₆ alkoxy group.

6. The compound for use according to any one of claims 2 to 5, wherein
R₃ is hydrogen, or a C₁₋₆ alkyl group; and
R₄ is hydrogen, or a C₁₋₆ alkyl group.

7. The compound for use according to any one of claims 2 to 5, wherein
R₃ and R₄ are joined together to form a 5- or 6-membered monocyclic heterocyclyl group or a 9- or 10-membered bicyclic heterocyclyl group, each of which is optionally substituted by 1 to 3 substituents independently selected from;
(i) —NH₂;
(ii) —OH;
(iii) a halogen atom;
(iv) a C₁₋₆ alkyl group;
(v) a C₁₋₆ alkoxy group;
(vi) a C₆₋₁₀ aryl group optionally substituted by 1 to 3 substituents independently selected from: a halogen atom and a group of the formula —SO₂R²², wherein R²² is hydrogen, a C₁₋₃ alkyl group, a group of the formula —NR²³R²⁴, wherein R²³ and R²⁴ are each individually selected from hydrogen and a C₁₋₃ alkyl group, or a 6-membered heterocyclyl group optionally substituted by a C₁₋₃ alkyl group.

8. The compound for use according to any one of claims 2 to 4 and 6, wherein the compound is a compound of the formula: 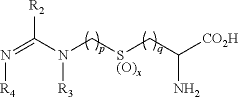
wherein
R₂ is a C₁₋₆ alkyl group, a C₂₋₆ alkenyl group, a C₂₋₆ alkynyl group, or a C₃₋₆ cycloalkyl-C₁₋₆ alkyl group, each of which is optionally substituted by 1 to 3 substituents independently selected from:
(i) —CN;
(ii) —NO₂;
(iii) a group of the formula —COR⁸, wherein R⁸ is hydrogen, a C₁₋₆ alkyl group, a group of the formula —OR⁹, wherein R⁹ is hydrogen or C₁₋₆ alkyl, or a group of the formula NR¹⁰R¹¹, wherein R¹⁰ and R¹¹ are independently selected from hydrogen or a C₁₋₆ alkyl group;
(iv) a group of the formula —S(O)ₘR¹², wherein m is 0, 1 or 2, R¹² is hydrogen, a C₁₋₆ alkyl group, hydroxy or a group of the formula NR¹³R¹⁴ wherein R¹³ and R¹⁴ are independently hydrogen or a C₁₋₆ alkyl group; (v) a group of the formula PO(OR¹⁵)₂, wherein R¹⁵ is hydrogen or a C₁₋₆ alkyl group;
(vi) a group of the formula NR¹⁶R¹⁷, wherein R¹⁶ and R¹⁷ are independently selected from hydrogen, a C₁₋₆ alkyl group, a group of the formula —COR¹⁸, wherein R¹⁸ is hydrogen or a C₁₋₆ alkyl group, or a group of the formula —S(O)_{m′}R¹⁹, wherein m′ is 0, 1 or 2 and R¹⁹ is hydrogen or a C₁₋₆ alkyl group;
(vii) a halogen atom; and
(viii) a group of the formula —OR²⁰, wherein R²⁰ is hydrogen, a C₁₋₆ alkyl group optionally substituted by 1 to 3 halogen atoms, a C₆₋₁₀ aryl group or a group of the formula —COR²¹, wherein R²¹ is hydrogen or a C₁₋₆ alkyl group;
R₃ and R₄ are each hydrogen;
p is 2 or 3;
q is 1 or 2; and
x is 1 or 2.

9. The compound for use according to claim 2, wherein the compound is selected from: 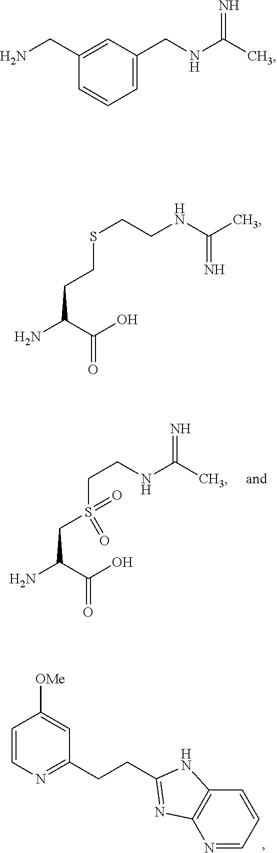
including pharmaceutically acceptable salts thereof.

10. The compound for use according to claim 1, wherein the compound has a structure according to:
Formula (I) of WO 95/34534;
Formula (I) of WO 2005/030768;
Formula (I) of WO 2005/030770;
Formula (I) of WO 2007/039578;
Formula (I) of WO 2007/045622;
Formula (I) of WO 2005/030769;
Formula (I) of WO 2005/030771;
Formula (I) of WO 03/080607;
Formula (I) of WO 2005/061496;
Formula (I) of WO 2005/026143;
Formula (I) of WO 2008/031788;
Formula (I) of WO 2006/103255;
Formula (I), (II) or (III) of WO 03/092678;
Formula (Ya), (Yb), or (Yc) of WO 01/14371;
Formula (I) of US 2003/0069210;
Formula 1 of WO 2008/072937;
Formula 1 of WO 01/72703;
Formula 1 of WO 01/72702;
Formula I of WO 02/22559;
Formula I of WO 2005/025620;
Formula I of WO 02/22562;
Formula (I) of WO 91/13055;
Formula (I) of WO 95/25717;
Formula (II) of WO 2006/060424;
Formula (I) of WO 2007/062410;
Formula (I) of WO 2007/062417;
Formula (I) of WO 2007/117778;
Formula (I) of WO 2008/103615;
Formula I or II of WO 2009/029625
Formula I of WO 2007/084868;
Formula I or VI of WO 2007/101213;
Formula (I) of WO 2004/041794;
Formula (I) of WO 2004/009580;
Formula (I) of WO 03/01183 1;
Formula (I) of WO 03/029185;
Formula (I) of WO 2004/009579,
Formula (III) of CN 102702298,
Formula 2 of Schulz et al., Bioorg. Med. Chem., 2013, 21 (17), 5518-5531,
Formula (II) of WO 2006/060424,
Formula (II) of WO 2009/029617,
Formula (I) of WO 2005/030768,
Compounds 1 and 2 of Lee at al., J. Nat. Prod. 2014, 77 (6), 1528-1531,
Compounds 3a-g of Stefani et al., Euro. J. Med. Chem. 2012, 58, 117-127, or
Compounds 1-158 of Suaifan et al., J. Mol. Graph. Model. 2012, 37, 1-26, including pharmaceutically acceptable salts thereof.

11. The compound for use according to claim 1, wherein the compound has a structure selected from Table 3, including pharmaceutically acceptable salts thereof.

12. The compound for use according to any preceding claim, wherein the virus is rhinovirus, influenza virus (A and B), parainfluenza virus (1, 2 and 3), respiratory syncytial virus, adenovirus, coronavirus (e.g. SARS coronavirus), avian flu, Epstein-Barr virus, enterovirus, metapneumovirus, adenovirus, measles virus, herpes simplex virus, varicella-zoster virus, ebola virus or cytomegalovirus.

13. The compound for use according to any preceding claim, wherein the viral infection causes a condition selected from influenza, pharyngitis, rhinopharyngitis, laryngitis, gingivostomatitis, parotitis, pneumonia, bronchitis, bronchiolitis, laryngotracheobronchitis, rhinitis, sinusitis, tonsillitis, tracheitis, measles, chicken pox, asthma exacerbation, chronic obstructive pulmonary disease exacerbation, bronchiectasis exacerbation, cystic fibrosis exacerbation, otitis media, viral-associated wheeze, and severe acute respiratory syndrome.

14. A method of preventing viral replication and/or preventing or treating viral infections in a subject comprising administering a prophylactically or therapeutically effective amount of a compound as defined in any one of claims 1 to 11.
